(19) United States
(12) Patent Application Publication
(10) Pub. No.: US 2022/0249740 A1
(43) Pub. Date: Aug. 11, 2022

(54) BIOMIMETIC TISSUE AND METHOD OF USE THEREOF

(71) Applicants: New York Stem Cell Foundation, Inc., New York, NY (US); New York University, New York, NY (US)

(72) Inventors: Giuseppe Maria de Peppo, New York, NY (US); Elisa Riedo, Tenafly, NJ (US)

(21) Appl. No.: 17/592,169

(22) Filed: Feb. 3, 2022

Related U.S. Application Data
(60) Provisional application No. 63/145,212, filed on Feb. 3, 2021.

(51) Int. Cl. A61L 27/36; A61L 27/16; A61L 27/54; C12N 5/00; G01N 33/50; B33Y 10/00; B33Y 70/00

(52) U.S. Cl. CPC A61L 27/3691; A61L 27/16; A61L 27/54; C12N 5/0062; C12N 2533/30; G01N 33/5088; B33Y 10/00; B33Y 70/00; C12N 2513/00; C12N 5/0068

(57) ABSTRACT

Described herein are methods for using localized source of heat, such as thermal scanning probe lithography (tSPL), for the low-cost and high-throughput fabrication of biological tissue replicas.

### CROSS REFERENCE TO RELATED APPLICATIONS

This application claims priority to U.S. Provisional Application No. 63/145,212, filed Feb. 3, 2021, which is hereby incorporated by reference herein in its entirety.

### BACKGROUND OF THE INVENTION

The ability to replicate the microenvironment of biological tissues creates unique biomedical possibilities for stem cell applications. Current fabrication methods are limited by either the control on feature size and shape, or by the throughput and size of the replicas.

Thus, there is a need in the art for improved fabrication methods of biomimetic materials. This invention satisfies this unmet need.

### SUMMARY OF THE INVENTION

In one aspect, the present invention provides a method of producing a biological tissue replica comprising: using a localized source of heat to pattern and replicate the morphology of a biological tissue in a thermosensitive polymer coating a substrate, and wherein the thermosensitive polymer is biocompatible or cell culture compatible, thereby producing the biological tissue replica.

In one embodiment, the method comprises using thermal scanning probe lithography (tSPL) to pattern and replicate the morphology of the biological tissue in the thermosensitive polymer coating the substrate. In one embodiment, tSPL is conducted with a dwell time of about 40-70 μs and a pixel size of about 12-20 nm.

In one embodiment, the substrate comprises a solid substrate, a combination of materials, or a stacked combination of materials. In one embodiment, the substate comprises glass, quartz, silicon, metal, or ceramics. In one embodiment, the substrate is a medical device. In one embodiment, the medical device is an orthopedic implant or dental implant.

In one embodiment, the thermosensitive polymer has a stiffness that can be tuned by heat or UV light, with values ranging from kPa to GPa. In one embodiment, the stiffness of the thermosensitive polymer can be spatially patterned with micron scale resolution. In one embodiment, the thermosensitive polymer is polymethacrylate-carbamate-cinnamate copolymer (PMCC).

In one embodiment, the replicated morphology is further transferred using one or more etching procedures from the thermosensitive polymer to the substrate.

In one embodiment, the biological tissue replica has a nanometer resolution.

In one embodiment, an atomic force microscopy image, scanning electron microscopy image, and/or transmission electron microscopy image of the biological tissue morphology is used as an input for the patterning. In one embodiment, the image is filtered by setting a threshold value to separate the image into background pixels and foreground pixels, wherein the background pixels are not patterned and wherein the foreground pixels are patterned.

In one embodiment, the method comprises periodically washing the thermal probe during the patterning.

In one embodiment, the method comprises exposing amine groups of the polymer and attaching a biomolecule to the exposed amine groups to biofunctionalize the patterned morphology.

In one embodiment, the method comprises seeding cells onto the biological tissue replica and culturing the cells in a cell culture media. In one embodiment, the cells are selected from the group consisting of: stem cells, progenitor cells, mesenchymal stem cells, induced mesenchymal stem cells, induced pluripotent stem cells, embryonic stem cells, osteocytes, osteoblasts, osteoclasts, osteoprogenitor cells, mesenchymal cells, chondrocytes, cartilage progenitor cells, fibroblasts, endothelial cells, myocytes, cardiomyocytes, cardiac progenitor cells myoblasts, skin cells, skin stem cells, and tumor cells.

In one aspect, the present invention provides a biological tissue replica comprising a patterned biological tissue morphology on a substrate coated with a thermosensitive polymer to replicate the biological tissue morphology, wherein the thermosensitive polymer is biocompatible or cell culture compatible. In one embodiment, the replica is made by using thermal scanning probe lithography (tSPL) to pattern a biological tissue morphology on the substrate. In one embodiment, the patterned morphology has nanometer sized resolution. In one embodiment, the replica comprises cells seeded on the replica.

In one aspect, the present invention provides a method of performing an assay comprising: a) seeding a biological tissue replica with cells; b) culturing the cells in a cell culture media; and c) performing an assay using the cultured cells, thereby performing the assay.

In one aspect, the present invention provides a method of high throughput thermal scanning probe lithography (tSPL) for generation of a tissue replica, comprising: a) obtaining a digital image of a tissue; b) identifying background pixels and foreground pixels in the image; c) removing the background pixels to produce a filtered image of the tissue; d) obtaining a transparent glass substrate coated with polymethacrylate-carbamate-cinnamate copolymer (PMCC); and patterning the filtered image into the PMCC coating to form a tissue replica.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1a-FIG. 1i: Schematic representation of bio-tSPL to produce mm size bone tissue replicas. FIG. 1a) Structure of human cortical bone composed of twisted plywood arranged fibers containing collagen fibrils, showing the characteristic 67 nm periodicity. FIG. 1b) AFM amplitude image of the bone tissue. FIG. 1c) Processing of the bone tissue image through threshold filtering to increase throughput. FIG. 1d) Patterning of large-scale bone tissue replicas on a pristine polymer resist using the filtered input image in (FIG. 1c) and optimized writing parameters. FIG. 1e) Halting patterning and cleaning the spoiled thermal probe with chloroform. FIG. 1f) Continuing patterning of large-scale bone tissue replicas on a pristine polymer film. FIG. 1g) Cell culture on the large-scale bone tissue replicas. FIG. 1h) Cell removal and sample washing. FIG. 1i) Reuse of the large-scale bone tissue replicas for cell culture studies.

FIG. 2a-FIG. 2p: Cell culture compatibility of the PMCC resist. FIG. 2a-FIG. 2d) Light micrographs of human iMSCs attaching to the PMCC resist (FIG. 2a) and (FIG. 2b), and tissue culture plastic (TCP) (FIG. 2c) and (FIG. 2d) 2.5 and 5 h after seeding, respectively. FIG. 2e and FIG. 2f) Fluorescence micrographs of human iMSCs attached to the PMCC resist (FIG. 2e) and TCP (FIG. 2f) 1 day after seeding. FIG. 2g) SEM images of human iMSCs attached to the PMCC resist 1 d after seeding. FIG. 2h) Metabolic activity of human iMSCs growing on the PMCC resist and TCP 1 d after seeding. FIG. 2i and FIG. 2j) Light images of human iMSCs grown onto the PMCC resist and TCP one week after seeding. k-n) Confocal fluorescence images showing live (green) and dead (red) cells on the PMCC resist and TCP after one week of culture. FIG. 2o) Percentage of viable cells on the PMCC resist and TCP as measured by quantification of LIVE/DEAD images in (FIG. 2k)-(FIG. 2n) (black histogram) and using the acridine orange and propidium iodide viability assay (white histogram). FIG. 2p) Metabolic activity of human iMSCs growing on the PMCC resist and TCP after 1 week of culture. Scale bar 50 μm for (FIG. 2a-FIG. 2f), 5 μm for (FIG. 2g), and 100 μm for (FIG. 2i-FIG. 2n).

FIG. 3a-FIG. 3q: Strategy for scaling up tSPL patterning. FIG. 3a) AFM amplitude error image of the bone tissue microenvironment used as an input template for tSPL patterning. FIG. 3b) Large scale input template generated by repeating the input image in (FIG. 3a) in a 15×15 square array. The black frame shows the area that can be patterned without changing thermal probe, i.e., 181 μm² corresponding to 20 bone images as in (FIG. 3a). FIG. 3c-FIG. 3e) In situ tSPL thermal images of the 1st, 10th, and 20th tSPL bone tissue replicas fabricated in the PMCC resist. f) Image obtained by threshold filtering of the image in (FIG. 3a), used as new input image for tSPL patterning. FIG. 3g) Large scale input template generated by repeating the filtered input image in (FIG. 3f) in a 15×15 square array. The red frame shows the area that can be patterned without changing thermal probe, i.e., 1076 μm² corresponding to 119 bone images as in (FIG. 3f). FIG. 3h-FIG. 3l) In situ tSPL thermal images of the 1st, 67th, and 119th tSPL bone tissue replicas fabricated in the PMCC resist, showing the enhanced probe durability when the filtered input image is used. FIG. 3k) Depth and D-spacing of the patterned collagen fibril nanostructures (FIG. 1a) versus patterning area for input arrays in (FIG. 3b) (black), and (FIG. 3g) (red). Inset: zoom-in of the green frame in (FIG. 3c), with corresponding cross-sectional profile of a patterned collagen fibril. FIG. 3l and FIG. 3m) tSPL software-estimated time needed to pattern one single filtered input image as in (FIG. 3f), as a function of dwell time and pixel size. FIG. 3n-FIG. 3q) tSPL bone tissue replicas of FIG. 3f) in PMCC for 14 to 20 nm per pixel size. Inset: Fourier analysis of the respective replicas. Scale bar: 1 μm in all images.

FIG. 4a-FIG. 4g: Fidelity of the bone tissue replica and tSPL patterning resolution. FIG. 4a) In situ tSPL thermal image of a tSPL bone tissue replica fabricated in the PMCC resist. Scale bar: 1 μm. b) Cross-sectional profiles of two segments shown in (FIG. 4a), highlighting the presence of nm gaps and twin-peak fibril bumps, comparable to the structure of a native type I collagen fibril, see SEM image in the inset. Reproduced with permission (J. F. W. Greiner, et al., Nanomed. Nanotechnol. 2019, 17, 319) Copyright 2020, Elsevier (RightsLink). FIG. 4c) Cross-sectional profile (blue line) of a replicated fibril in (a) and comparison with a cross-sectional profile (black line in inset) of a native type I collagen fibril (from S. Bansode, et al., Sci. Rep. 2020, 10, 3397), showing the same characteristic 67 nm periodicity. FIG. 4d and FIG. 4e) Cross-sectional profiles of three gaps (FIG. 4d) and bumps (FIG. 4e) in the replicated fibrils in (FIG. 4a). FIG. 4f) In situ tSPL thermal image of parallel line-patterns in the PMCC resist. Scale bar: 100 nm. FIG. 4g) Corresponding cross-sectional profile of the line-cut in (FIG. 4f) featuring sub-10 nm FWHM, 11 nm half pitch, and 2 nm resolution in the z-direction.

FIG. 5a-FIG. 5k. Large scale tSPL bone tissue replicas for cell culture studies. FIG. 5a) Optical image of a tSPL large scale bone tissue replica (0.5 mm×0.5 mm) patterned in the PMCC resist spin coated on ITO glass. Scale bar: 100 μm. FIG. 5b) AFM image of part of the bone tissue replica shown in (FIG. 5a). Scale bar: 5 μm. FIG. 5c) High-magnification zoom-in AFM topography image of one replica unit in (FIG. 5b). Scale bar: 1 μm. FIG. 5d) Light microscopy image of a large-scale bone tissue replica (indicated with a white frame) before cell culture. FIG. 5e and FIG. 5f) Light microscopy image of human iMSCs growing on the bone tissue replica. Scale bar: 50 μm. FIG. 5g) Number of cells grown after three days on the unpatterned and patterned (bone replica) area. FIG. 5h-FIG. 5k) Confocal images of human iMSCs growing on the bone tissue replica and stained for F-actin, vinculin, and cell nuclei. Scale bar: 50 μm.

FIG. 6a-FIG. 6j. Reusability of the PMCC resist for repeated cell culture studies. FIG. 6a) AFM topography image of several grooves patterned in pristine PMCC resist spin coated on ITO glass. Scale bar: 1 μm. FIG. 6b-FIG. 6h) Light microscopy images of human iMSCs cultured on the same PMCC resist sample during seven consecutive culture cycles. Scale bar: 50 μm. At the end of each culture experiment, cells are detached by incubation with trypsin. Insets show the corresponding AFM topography images of the same pattern in FIG. 6a after each cell culture, cell removal, and sample washing cycle. All AFM images have a z-scale of 38 nm. Scale bar: 1 FIG. 6i) Average cross-sectional profiles of the grooves shown in (FIG. 6a) and in the insets of (FIG. 6b)-(FIG. 6h). FIG. 6j) Average values for groove depth (dᵢ) and full width at half maximum (wᵢ), where i is the index for different experiment cycles.

FIG. 7a-FIG. 7g: Simultaneous patterning of bone tissue topography and amine chemistry. FIG. 7a-FIG. 7c) Schematic illustration of the tSPL patterning process and pattern biofunctionalization where (FIG. 7a) is the PMCC carbamate block chemical structure showing protected amine, (FIG. 7b) the thermal exposure of amine, (FIG. 7c) the biofunctionalization of the amine pattern. FIG. 7d) Fluorescence microscopy image of an array of bone tissue replicas patterned using different probe temperatures and functionalized with Alexa 488 dye. Scale bar: 5 μm. FIG. 7e-FIG. 7g) In situ thermal topography images of individual tSPL replicas patterned with different probe temperatures. Scale bar: 1 μm. Each image corresponds to the corresponding color coded dash-framed fluorescent pattern in (FIG. 7d).

FIG. 8: Culture of human iMSCs on the PMCC resist and patterned replica. Light micrographs at different magnification showing the cells growing on the PMCC resist and on the patterned replica identified by a white rectangular frame. Scale bar: 200 μm.

FIG. 9a-FIG. 9f: Comparison between tSPL bone tissue replica (blue frame) and bone tissue reproduced from references (red frame). (FIG. 9a) In situ tSPL thermal images of a tSPL bone tissue replica fabricated in the PMCC resist. Scale bar: 1 μm. (FIG. 9b) Cross-sectional profile (blue line) of a replicated fibril in (FIG. 9a) showing the same characteristic 67 nm periodicity. (FIG. 9c) Cross-sectional profile of two segments of the bone tissue replica shown in FIG. 4a, highlighting the presence of ˜20-30 nm gaps and twin-peaks collagen bumps. (FIG. 9d) AFM topography height image of a type I collagen fibril from Bansode et al., Sci Rep, 2020, 10 (FIG. 9e) Corresponding cross-sectional profile of the collagen fibril in (FIG. 9d). (FIG. 9f-D) SEM image of the cross-striated pattern of collagen fibrils with pore-like structures of approximately 30±5 nm (arrows) from Greiner et al., Nanomed-Nanotechnol, 2019, 17, 319 (f-E) SEM image (FIG. 9f-E-a) and FFT-analysis (FIG. 9f-E-b) of the pore structure of a collagen fibril revealing distinct pore sizes of 31.93±0.97 nm. (FIG. 9f-F) Scheme of the 30 nm gap region of collagen single repeating segment. Data in (FIG. 9d and FIG. 9f) were reproduced from Bansode et al., Sci Rep, 2020, 10. (Creative Commons Attribution 4.0 International License with permission of Springer Nature). Data in (FIG. 9f) were Reproduced from Greiner et al., Nanomed-Nanotechnol, 2019, 17, 319, copyright 2020, with permission of Elsevier (RightsLink).

FIG. 10a-FIG. 10h: Thermal probe cleaning. (FIG. 10a-FIG. 10b) tSPL thermal images of a single-line patterned by a soiled probe (FIG. 10a) and by the same probe after cleaning (FIG. 10b) in chloroform. Z scale: 13 nm. Scale bar: 200 nm. (FIG. 10c) Cross-sectional profiles of the patterned lines in (FIG. 10a) and (FIG. 10b). (FIG. 10d) FWHM of single lines patterned by three probes before and after cleaning. (FIG. 10e-FIG. 10h) tSPL thermal images of a bone tissue replica patterned by the same probe successively (note that (FIG. 10h) was patterned after cleaning in chloroform). Z scale: 26 nm. Scale bar: 1 μm.

FIG. 11a-FIG. 11g: tSPL thermal images at different dwell-time. (FIG. 11a-FIG. 11f) tSPL bone tissue replicas patterned in the PMCC resist using 16 nm pixel size and different dwell-times (40-150 μs). (FIG. 11g) Depth and D-spacing as a function of dwell-time. Scale bar 1 μm.

FIG. 12a-FIG. 12e: Writing process adjustment for throughput enhancement. FIG. 12a) Schematic illustration of the tSPL probe trajectory for patterning a single bone tissue replica unit. Each turnaround per line takes about 0.05 s. FIG. 12b) An example of the patterned bone tissue replica unit resulting from the patterning process illustrated in (FIG. 12a). FIG. 12c) Schematic illustration of patterning 20 bone tissue replica units (60 μm in length) one by one in a row using the method in (FIG. 12a). This takes 20 turnovers per line. FIG. 12d) Schematic illustration of patterning the same 20 bone tissue replica units all together in a row in one patterning process. This takes only 1 turnover per line. FIG. 12e) An example of 18 bone tissue replicas in a row resulting from the patterning method depicted in (FIG. 12d). The input images and thermal topography images of the bone tissue replica units are 3.0 μm×2.5 μm in size.

FIG. 13a-FIG. 13b: Thermal probe durability. FIG. 13a) Filtered array input image used as input template in the Nanofrazor Software. The red dotted line marks the area written with a single probe without probe changing/washing (15×7 replica units). FIG. 13b) AFM image of a region (5×4 replica units) of the bone tissue replica fabricated using the filtered input image in FIG. 13a where the 7th row is the last patterned with the same probe.

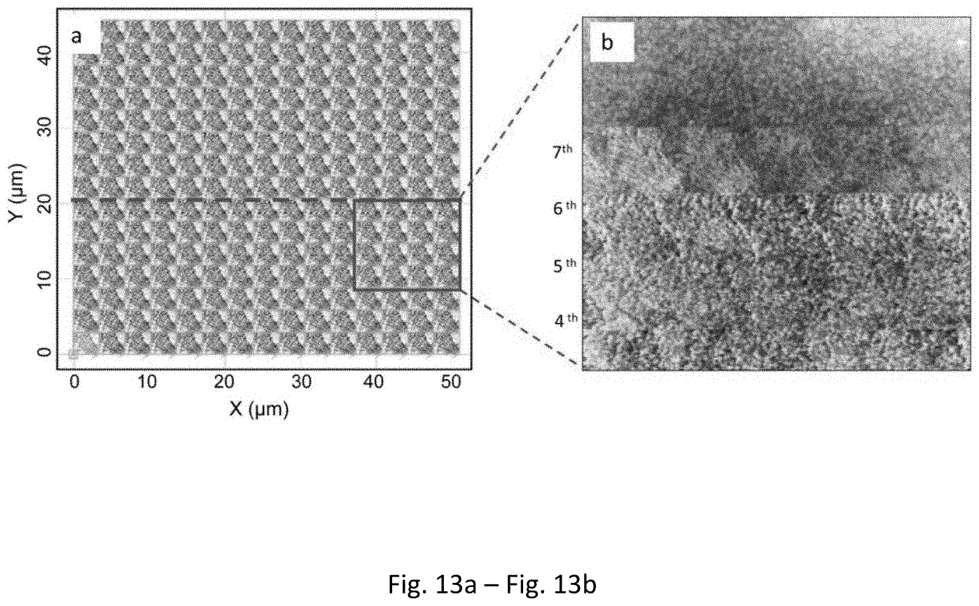

FIG. 14a-FIG. 14b: Cell growth on a patterned replica. FIG. 14a) Light microscopy image of human iMSCs growing on a bone tissue replica indicated by a white frame. FIG. 14b) Light microscopy image of the same region showing a cell crawling over the patterned area a few hours later. Scale bar: 100 μm.

FIG. 15 depicts an illustrative computer architecture for a computer 200 for practicing the various embodiments of the invention.

### DETAILED DESCRIPTION

The present disclosure describes a platform that utilizes localized heat to pattern and replicate the morphology of biological tissue in a thermosensitive polymer to produce a tissue replica.

For example, in one aspect, the present invention combines thermal scanning probe lithography (tSPL) with innovative methodologies for the low-cost and high-throughput nanofabrication of large area quasi-3D tissue replicas with high fidelity, sub-15 nm lateral precision, and sub-2 nm vertical resolution.

Accordingly, in one aspect, the invention provides a method of producing a tissue replica. The method includes patterning a tissue morphology on a substrate coated with a thermosensitive polymer, wherein the thermosensitive polymer is patterned to replicate the tissue morphology via tSPL, and wherein the thermosensitive polymer is cell culture compatible.

In another aspect, the invention provides a tissue replica. In one embodiment, the tissue replica is produced by the method of the invention.

In yet another aspect, the invention provides a method of performing an assay. The method includes: seeding the tissue replica of the invention with cells; culturing the cells in a cell culture media; and performing an assay using the cultured cells, thereby performing the assay.

### Definitions

Unless defined otherwise, all technical and scientific terms used herein have the same meaning as commonly understood by one of ordinary skill in the art to which this invention belongs.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present disclosure describes a platform that utilizes localized heat to pattern and replicate the morphology of biological tissue in a thermosensitive polymer to produce a tissue replica. For example, in one embodiment, the present disclosure relates to the use of tSPL to pattern and replicate tissue morphology of any biological tissue or biological compartment. The replicas can be generated in a thermosensitive polymer coating a substrate. In certain aspects the substrate is transparent allowing the replicas to be used a variety of experiments. In some embodiments, the substrate is a medical device, such as a orthopedic implant or dental implant, thereby allowing the replicas to support the growth of seeded cells on the medical device.

The present invention can be used to produce any types of tissue replicas of any type of biological tissue, including, but not limited to, bone tissue, muscle tissue, cardiac tissue, cartilage, skin, tumors and the like. The invention can also be used to produce replicas of any biological tissue compartment such as stem cell niches. Thus, while the data presented herein may exemplify the production of bone tissue replicas, a skilled artisan would recognize that the present invention can similarly be used to fabricate tissue replicas of any biological tissue or tissue compartment.

In the human body, cells reside in specific microenvironments consisting of an intricate network of extracellular matrix (ECM) components that support the cells and provide topographical, chemical, and mechanical cues regulating cell fate. In particular, in the bone tissue cells are embedded in a matrix of hierarchically organized collagen fibrils, bone specific glycoproteins, and hydroxyapatite crystals (J. W. Zhang, et al., Nature 2003, 425, 836; L. M. Calvi, et al., Nature 2003, 425, 841; T. Yin, et al., J. Clin. Invest. 2006, 116, 1195), having features size in the range of 10-300 nm (J. M. Wallace, et al., Langmuir 2010, 26, 7349; J. M. Wallace, et al., Bone 2010, 46, 1349)

Multiscale microenvironmental features, from micro- to nanoscale, influence cell morphology, migration, proliferation, and differentiation (M. M. Stevens, et al., Science 2005, 310, 1135; C. J. Bettinger, et al., Angew. Chem., Int. Ed. Engl. 2009, 48, 5406; A. Kumar, et al., Development 2017, 144, 4261). The ability to fabricate high fidelity replicas of the bone tissue microenvironment with nanoscale resolution creates the possibility to study the regulatory mechanisms governing the cell-ECM interaction process in vitro, and ultimately harness the regenerative capacity of stem cells and tissues for a variety of biomedical applications (H. Donnelly, et al., Nanomed. Nanotechnol. 2018, 14, 2455). At present, stem cell differentiation relies only on using a cocktail of molecules on standard plastic substrates, which do not provide the cells with the topographical, and mechanical signals present in the tissue microenvironment, and which are critical for controlling stem cell behavior and full differentiation into mature cell types (M. J. Dalby, et al., Nat. Mater. 2014, 13, 558; J. H. Wen, et al., Nat. Mater. 2014, 13, 979; D. E. Discher, et al., Science 2009, 324, 1673).

Over the past decades, bottom-up self-assembly methods have been extensively used to study the biological response and behavior of stem cells to specific topographies (G. M. de Peppo, et al., Int. J. Nanomed. 2014, 9, 2499; P. Y. Wang, et al., ACS Appl. Mater. Interfaces 2015, 7, 4979; S. Guven, et al., Trends Biotechnol. 2015, 33, 269). For example, self-assembly of oligopeptides containing cell adhesion motifs (C. S. Chen, et al., Science 1997, 276, 1425; S. G. Zhang, et al., Biomaterials 1999, 20, 1213), nanofibers alone (J. Hu, et al., Pharm. Res. 2011, 28, 1273; L. A. Smith, et al., Soft Matter 2008, 4, 2144), or combined with hydrogels (A. Aravamudhan, et al., RSC Adv. 2016, 6, 80851) have been used to fabricate surface patterns that mimic tissue characteristics and improve stem cell differentiation. However, self-assembly methods lack precise control of shape and size of the nanopatterned features and flexibility on the choice of the environment. On the other hand, the rapid development of top-down lithographic methods, such as photolithography (A. Goulet-Hanssens, et al., Biomacromolecules 2012, 13, 2958; J. Friguglietti, et al., Biomaterials 2020, 244, 119927; H. Kavand, et al., ACS Appl. Mater. Interfaces 2019, 11, 10559), electron beam lithography (M. J. Dalby, et al., Nat. Mater. 2007, 6, 997; C. P. W. Hammann, et al., Microelectron. Eng. 2018, 195, 13), soft lithography (J. Friguglietti, et al., Biomaterials 2020, 244, 119927; M. J. Li, et al., Biomaterials 2019, 216, 119269), and nanoimprinting lithography (M. Domanski, et al., Nanotechnology 2012, 23, 065306; H. R. Seo, et al., ACS Appl. Mater. Interfaces 2017, 9, 16804), has led to the fabrication of a variety of synthetic surfaces with well-defined but simple geometrical features, such as pillars, squares, bumps, and grooves (S. Gerecht, et al., Biomaterials 2007, 28, 4068; C. J. Bettinger, et al., Adv. Mater. 2008, 20, 99; A. I. Teixeira, et al., Cell Sci. 2003, 116, 1881). In general, bottom-up and top-down fabrication approaches are unable to reproduce the exact nano- and microscale morphology and chemistry of the bone tissue, and therefore are limited in their ability to produce biomimetic surfaces that accurately control cell behavior (K. Metavarayuth, et al., ACS Biomater. Sci. Eng. 2016, 2, 142).

In contrast, thermal scanning probe lithography (tSPL) (R. Szoszkiewicz, et al., Nano Lett. 2007, 7, 1064) has been proven to be capable of patterning complex quasi-3D topographies on a polymer surface with sub-15 nm lateral resolution and sub-2 nm depth resolution (D. Pires, et al., Science 2010, 328, 732; R. Garcia, et al., Nat. Nanotechnol. 2014, 9, 577; X. Y. Liu, et al., ACS Appl. Mater. Interfaces 2019, 11, 41780). Nowadays, tSPL can be performed using a commercially available instrument, which uses a thermal nanoprobe to locally evaporate the thermosensitive polymer polyphthalaldehyde (PPA), leaving a void that defines the pixel size (S. T. Zimmermann, et al., ACS Appl. Mater. Interfaces 2017, 9, 41454; S. T. Howell, et al., Microsyst. Nanoeng. 2020, 6, 21; X. R. Zheng, et al., Nat. Electron. 2019, 2, 17). While tSPL has found very promising applications in nanoelectronics and photonics (R. Garcia, et al., Nat. Nanotechnol. 2014, 9, 577; S. T. Howell, et al., Microsyst. Nanoeng. 2020, 6, 21; X. R. Zheng, et al., Nat. Electron. 2019, 2, 17; Z. Q. Wei, et al., Science 2010, 328, 1373; S. H. Chen, et al., Nano Lett. 2019, 19, 2092; C. Rawlings, et al., Nanotechnology 2018, 29, 505302; C. D. Rawlings, et al., Sci. Rep. 2017, 7, 16502; S. Ristic, et al., Nano-Patterning of Single-and bi-level Surface-Relief Gratings using a Commercial Thermal Scanning Probe Lithography System, 2015 Photonics North, Ottawa, ON 2015, doi.org/10.1109/PN.2015.7292463), its use in biomedical research is still in its infancy because of the limited patternable areas and throughput, and the lack of biocompatible materials suitable for cell culture studies. Typical areas addressed by the tSPL fabrication method in the sub-15 nm high-resolution mode, are in the range of 1-10 μm², smaller than the size of a cell. Indeed, currently the prohibitive cost and exorbitant amount of time required for large-scale tSPL patterning with sub-15 nm resolution makes the nanofabrication of millimeter size replicas of the tissue microenvironment impracticable. For example, researchers have recently replicated the nanoscale morphology of a biological tissue in a film of PPA polymer stabilized on a silicon wafer by an adhesive layer of allylamine (ALA) (S. W. Tang, et al., ACS Appl. Mater. Interfaces 2019, 11, 18988). Using this approach, they fabricated a generalized tendon pattern, inducing formation of focal adhesions similarly to native tendon sections. However, the produced replicas were only a few μm² in size, which hinders their potential for biomedical applications. Moreover, their strategy to replicate the tendon topography over larger areas does not enable the fabrication of a pattern recapitulating the exact 3D topography of biological tissues. Indeed, to fabricate using tSPL large-scale biological tissue replicas with faithful 3D topography, there are limiting factors related to low throughput and high cost including: (i) intrinsic scanning speed of the thermal probe, (ii) time to write each pixel, (iii) number of pixels, and (iv) thermal probe contamination and replacement.

The present invention relates to a new bio-tSPL platform that dramatically reduces the problems of throughput and high cost of tSPL. For example, the system and methods described herein utilize smart software and post-patterning procedures, as well as a biocompatible, functional polymer resist that is shown herein to withstand multiple cell culture cycles, allowing the reuse of the tissue replicas.

The presently described system and method allows for the replication of the complex quasi-3D morphology of biological tissue over millimeter scale areas, with sub-15 nm resolution in x-y, and sub-2 nm resolution in z-direction. As described herein, the millimeter replicas are fabricated with increased throughput and reduced cost such that their production becomes now feasible for a variety of applications, such as biomedical research.

tSPL can write topographical and chemical features in a thermosensitive polymer resist by local heating, thereby carving and chemically activating the surface with nanoscale precision (X. Y. Liu, et al., ACS Appl. Mater. Interfaces 2019, 11, 41780; D. B. Wang, et al., Adv. Funct. Mater. 2009, 19, 3696). The tSPL process involves first the pixilation of a reference input image, and then the replication of that image by assigning at each grey level of individual pixels a particular height level. For biomedical oriented applications, the main drawback of SPL techniques, including tSPL, is the limited throughput due to the serial writing process, the low writing speed, and the high cost associated with large scale patterning. Furthermore, it is of key importance to identify a substrate that is transparent, cell-compatible, and stable in a wet hydrolytic environment at 37° C.

Here, the present invention is based upon the development of several steps to enable the replication of biological tissue morphology on a substrate to produce tissue replicates. For example, in certain embodiments, the method allows for replication of the tissue morphology over large areas with nanometer resolution. In one embodiment, the method allows for sub-2 nm resolution in z-direction. In one embodiment, the method allows for sub-15 nm resolution in x-y direction. In one embodiment, the method comprises the use of localized scanning heat to pattern a biocompatible and/or cell culture compatible polymer resist coated on a substrate. In one embodiment, the polymer resist is spin-coated on the substrate. In one embodiment, the polymer resist comprises polymethacrylate-carbamate-cinnamate copolymer (PMCC). In one embodiment, the substrate comprises glass, such as ITO glass.

The present invention utilized localized heat to pattern a thermosensitive polymer in order to replicate biological tissue morphology. An exemplary system that uses localized heat is tSPL, which is described further below. However, the present invention is not limited to any particular methodology. Thus, a skilled artisan would recognize that any methodology that uses localized heat and/or scanning heat to pattern the polymer can be used.

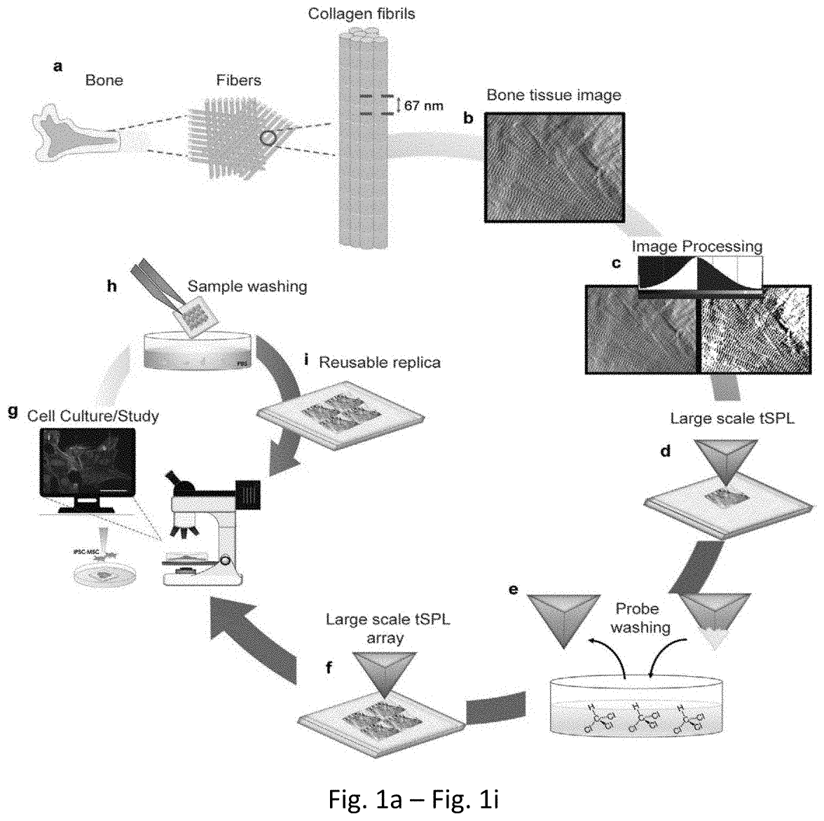

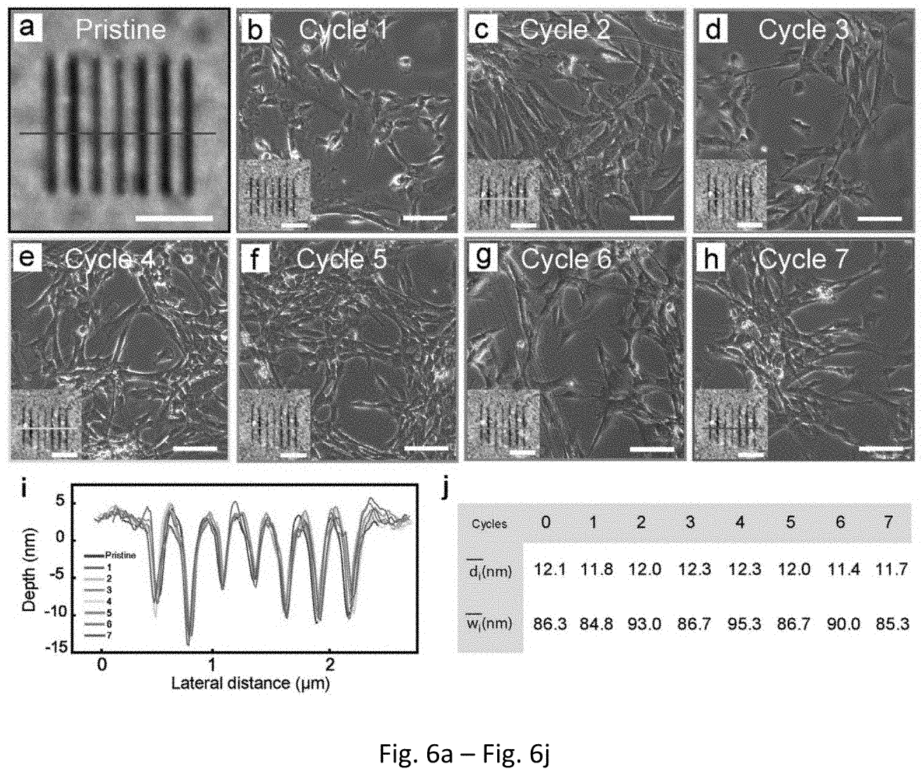

FIG. 1a-FIG. 1i provides a schematic diagram showing the exemplary steps of the presently described bio-tSPL process. In one embodiment, the method comprises inputting or uploading an atomic force microscopy (AFM) image bitmap input image of the tissue microenvironment in the tSPL software (FIG. 1a-FIG. 1b). In certain aspects, the method comprises applying a series of input image filtering processes (FIG. 1c), and patterning parameters optimization strategies to increase throughput and decrease cost. In one embodiment, the method comprises using tSPL to pattern tissue replicas in the biocompatible and/or cell culture compatible polymer resist (e.g., PMCC resist) (FIG. 1d). In one embodiment, the method comprises cleaning the thermal probes, which reduces the number of probes needed to generate large area replicates therefore reducing operational costs. In one embodiment, the method comprises culturing cells on the fabricated tissue replicas. (FIG. 1g). Further, as demonstrated herein, the fabricated tissue replicas are reusable for multiple rounds of cell culturing and experimentation, and thus, in certain embodiments, the method comprises removing the cells and cleaning the sample following each cell culture experiment (FIG. 1h-FIG. 1j and FIG. 6).

tSPL can write topographical and chemical features in a thermosensitive polymer resist by local heating, thereby carving and chemically activating the surface with nanoscale precision (X. Y. Liu, et al., ACS Appl. Mater. Interfaces 2019, 11, 41780; D. B. Wang, et al., Adv. Funct. Mater. 2009, 19, 3696). The tSPL process involves first the pixilation of a reference input image, and then the replication of that image by assigning at each grey level of individual pixels a particular height level. Additional details and description of tSPL can be found in U.S. Pat. No. 8,468,611 (where tSPL is referred to as thermochemical nanolithography), which is incorporated herein by reference in its entirety.

Generally, a tSPL system includes a probe (or a plurality of probes) and a resistive heater in electrical communication with the probe. In one embodiment, the probe is a silicon probe. In certain embodiments, the probe is an AFM instrument or AFM tip. For illustrative convenience, when referring herein to an AFM instrument itself (i.e., without an AFM tip attached thereto), it is intended that the AFM include any necessary constituents and equipment necessary to operate the AFM, as would be understood by those skilled in the art to which this disclosure pertains. Examples of such constituents include a probe head, a camera module, a piezoelectric scanner for embodiments where the surface that is patterned will move, optics to monitor the movement and position of the AFM tip, a device (if desired) to monitor atmospheric conditions (e.g., humidity, pressure, temperature, and the like), a chamber or other materials (if desired) to isolate the AFM from outside noise, and/or the like. Similarly, examples of such equipment for operating the AFM include a controller unit for controlling the AFM constituents, a computer with software for sending, receiving, and processing electronic signals to and from the controller and/or the AFM constituents, and/or the like.

Similarly, as used herein, the term “AFM tip” is intended to include both a cantilever and tip, which is located at the end of the cantilever, as would be understood by those skilled in the art to which this disclosure pertains. The AFM tip can be any type of silicon, silicon nitride, or other composition AFM tip known to those skilled in the art.

As stated above, the resistive heater is in electrical communication with the AFM tip. Specifically, the resistive heater can be physically coupled to the AFM tip (e.g., via the cantilever or cantilever holder of the AFM instrument), or it can comprise a portion of the AFM tip. In exemplary embodiments, the resistive heater comprises a portion of the AFM tip. Such AFM tips are known to those skilled in the art to which this disclosure pertains.

One type of AFM tip with an integrated resistive heater is described in Lee et al., “Electrical, Thermal, and Mechanical Characterization of Silicon Microcantilever Heaters,” Journal of Microelectromechanical Systems, 15, 1644 (2006), which is incorporated herein by reference as if fully set forth below. Briefly, these tips are made using a standard silicon-on-insulator (SOD process. The process starts with providing a SOI wafer having a <100> orientation, and n-type doping it at 2×10¹⁴ atoms per cubic centimeter (cm⁻³) to have a resistivity of about 4 ohm-centimeters (Ω-cm). The cantilever tip can be formed using an oxidation sharpening process such that it has a radius of curvature about 20 nm and a height of about 1.5 micrometers (μm). The cantilevers are made electrically active by selectively doping different parts of the cantilever through a two-step process. First, a low-dosage blanket ion implantation can be performed on the entire cantilever, followed by furnace-annealing in order to establish an essentially uniform background doping level (e.g., 10¹⁷ cm⁻³, phosphorous, n type). The cantilever then can be subjected to a heavy implantation step during which a region around the tip (e.g., having a width of about 8 μm) is masked off (10²⁰ cm⁻³, phosphorous n type). The masked region serves as a relatively lightly-doped region at the free end of the cantilever. It is this lightly-doped region that functions as the resistive heater. Finally, the cantilever can be electrically connected to the base via highly conducting legs (e.g., having a length of about 110 μm and a width of about 15 μm). With the cantilever dimensions and temperature-dependent resistivity, the resistive heater portion can account for more than about 90% of the electrical resistance of entire cantilever.

In some cases, the probe (e.g., AFM tip), regardless of how the resistive heater is placed in electrical communication thereto, can require modification of the original/standard cantilever- or chip-holder of the AFM instrument in order to provide current to heat the AFM tip. One such modification can include creating an electrical pathway or circuit for current to be applied (e.g., from a power source) to the AFM tip in a specific direction. This can be as simple as providing electrical leads to each side of the cantilever. If it is desirable to monitor the current applied to the AFM tip (e.g., for greater control of the heat generated at the AFM tip), then a voltmeter, multimeter, or like device can be included as part of electrical circuit. Further, if additional protective measures for the AFM tip are desired, then a sense resistor or like device can be placed in series with the AFM tip to limit the current applied to the AFM tip. These optional additional modifications can be useful for monitoring and/or controlling the temperature of the AFM tip during the tSPL process.

Once the system is fabricated or constructed, it can be used to pattern a surface. Such a process generally involves resistively heating the probe to a desired temperature, positioning the resistively heated probe adjacent to, or in contact with, a first location on a surface effective to heat the first location, and chemically modifying at least a portion of the first location. In certain cases, particularly when the probe is brought into contact with the surface, the chemical modification can also be a topographical modification.

The process can be repeated by discontinuing the positioning (i.e., removing the probe away from the first location on the surface), and re-positioning the heated probe with the surface at a second location so as to generate another chemical modification at the second location. This allows for multiple discrete locations on the surface to be patterned.

Alternatively, the process can be continued by moving the probe to a second location on the surface, while maintaining continuous proximity or contact between the heated probe and the surface from the first location all the way to the second location. This allows for a continuous pattern (i.e., chemical modification) to form on the surface from the first location through and to the second location. In this manner, both one- and two-dimensional patterns can be formed on the surface.

The cause of the chemical modification is the heat that is transferred from the probe to the material of the surface. Too little heat can result in no chemical modification, too much heat can result in excessive chemical modification (e.g., from thermal transfer beyond the area of proximity or contact between the probe and the surface, from additional chemical modification or even thermal decomposition of the surface, or both), and inconsistent heat can result in unintended patterning of the surface. Thus, in exemplary embodiments, a temperature calibration process is performed on the probe prior to initiating the tSPL process. The probes can be calibrated using thermometry techniques including optical thermometry micro-infrared thermometry, Raman spectroscopy, or the like. That is, these techniques can be used to measure the temperature of the probe at different electrical resistances or power levels in order to find the appropriate resistance or power level needed for a particular chemical modification.

In some cases, depending on the location of the resistive heater, there can be a temperature gradient in the probe itself. Thus, such calibration techniques can be used map the temperature profile of the entire probe.

In addition to the temperature of the probe, the amount of heat transferred to the surface can be influenced by the pressure applied by the probe to the surface. Thus, in exemplary embodiments, the spring constant of the probe is also calibrated. There are different methods for determining the spring constant of the probe, depending on its shape or geometry. Such methods are known to those skilled in the art to which this disclosure pertains. Additional information on calibration techniques can be found in the following references, which are incorporated herein in their entireties as if fully set forth below: Cleveland et al., “A nondestructive method for determining the spring constant of cantilevers for scanning force microscopy,” Review of Scientific Instruments, 64, 403 (1993); Hutter et al., “Calibration of atomic-force microscope tips,” Review of Scientific Instruments, 64, 1868 (1993); Sader et al., “Calibration of rectangular atomic force microscope cantilevers,” Review of Scientific Instruments, 70, 3967 (1999); Gibson et al., “Determination of the spring constants of probes for force microscopy/spectroscopy,” Nanotechnology, 7, 259 (1996); and Gibson et al., “A nondestructive technique for determining the spring constant of atomic force microscope cantilevers,” Review of Scientific Instruments, 72, 2340 (2001).

Yet another feature that can influence the amount of heat transferred to the surface is the exposure time itself. For embodiments where patterning speed is of importance, the probe will spend less time in a particular location and, therefore, less opportunity to effect complete thermal transfer from the probe to the surface if the probe is heated to the exact temperature needed to initiate the chemical modification. Thus, in these cases, those skilled in the art will recognize that probe should be heated to a temperature greater than the minimum chemical modification temperature in order to ensure sufficient thermal transfer for the desired chemical modification to occur. The extent to which the temperature of the probe exceeds the minimum chemical modification temperature will depend on the positioning or exposure time. That is, in certain instances, shorter exposure times will require greater temperatures in order to produce the same level of chemical modification as greater exposure times with lower temperatures, assuming that the probe is kept the same distance from the surface or (in cases where contact is made) that the pressure of the probe on the surface is kept the same.

The extent to which heat is transferred from the probe to the material of the surface will influence the resolution of the pattern. Thus, in addition to the general probe geometry, each of the factors listed above will affect both the thickness/fineness of a particular patterned shape and the density of patterned shapes that can be created in a given area of the surface.

Turning now to the surface itself, there are a variety of compositions that can be used to form the surface that is patterned or modified. In fact, any composition that can undergo a chemical reaction initiated by heat can be used to form the surface. Among the localized changes in the material that forms the surface that can be induced by the chemical reaction are one or more of the local elastic, mechanical, tribological, optical, wetting, adhesive, electrical, or chemical properties.

In general, the surface can be a liquid or solid. When the surface is a liquid, it can be placed in a container or vessel before being patterned using tSPL. As a solid, the surface can be a discrete body, or it can be disposed upon another material (e.g., a platform/substrate that can provide greater mechanical stability, for example, if the surface material itself is highly, thin or flexible).

In some embodiments, the surface is formed from a polymeric material. In general, the surface can be formed from polymers having the basic structure Pₙ-Gₙ, wherein P represents the polymer backbone, G represents a functional group that will be modified by tSPL, and n is a positive integer. The functional group, G, can form part of a polymer side-chain, or can be part of the polymer backbone. It should be noted that there can be more than one polymer backbone and/or functional group in the chosen polymer. Before tSPL, the polymer can be formed into a film using standard film-forming techniques such as spin-coating, drop casting, blade coating, and spray coating onto a substrate or platform. If desired, the substrate can be removed before subjecting the polymer film to tSPL.

In some embodiments, the polymer backbone, P, can be derived from, or can be, a monomer such as vinyl, allyl, 4-styryl, acroyl, epoxide, oxetane, cyclic-carbonate, methacroyl, acrylonitrile, or the like, which is polymerized by either a radical-, cationic-, atom transfer-, or anionic-polymerization process. In other cases, P can be derived from, or can be, an isocyanate, isothiocyanate, or epoxide, that can be copolymerized with di-functional amines or alcohols such as HO(CH₂)_{λ}OH, H₂N(CH₂)_{λ}NH₂, where λ is a positive integer (e.g., from 1 to 25). In other situations, P can be derived from, or can be, a strained ring olefin (e.g., dicyclopentadienyl, norbornenyl, cyclobutenyl, or the like), which can be polymerized via ring opening metathesis polymerization using an appropriate metal catalyst, as would be known by those skilled in the art to which this disclosure pertains. In still other embodiments, P can be derived from, or can be, (—CH₂)_{η}SiCl₃, (—CH₂)_{η}Si(OCH₂CH₃)₃, or (—CH₂)_{η}Si(OCH₃)₃, where the monomers can be reacted with water under conditions known to those skilled in the art to form either thin film or monolithic organically modified sol-gel glasses, or modified silicated surfaces, where η is a positive integer (e.g., from 1 to 25). Still further, P can be derived from, or can be, a polymerizable group that can be photochemically dimerized or polymerized.

In some embodiments, the functional group, G, can be chosen such that, upon heating from the probe, a protecting group is removed from the surface, leaving behind another functional group. For example, to obtain a carboxylic acid, G can be chosen form tert-butyl esters, tetrahydropyran esters, and the like. For an amine to result, G can include tetrahydropyranyl carbamates, amine N-oxides, and the like. If an alcohol or phenol is desired, G can be chosen from tetrahydropyranyl ethers, triphenylmethyl ethers, tetrahydropyranyl carbonate esters, and the like. When a thiol is desired after tSPL, G can include S-tert-butoxy carbonyls, S-tetrahydropyranyl carbonyls, ethyl disulfides, and the like.

In other cases, G can be a group that undergoes thermal polymerization and cross-linking reactions, including Diels-Alder reactions between two G groups (e.g., furans with maleimides, and the like), ring-opening polymerization (e.g., poly(ferrocenylsilanes) and the like), ring-opening metathesis polymerization (e.g., dicyclopentadiene, and the like), reactions to form conjugated polymers (e.g., from poly(phenylene-vinylene) or other like precursors), and reactions of trifluorovinyl ethers, for example. In still other situations, G can be a group that volatilizes or decomposes from the heat of the probe.

As stated above, the polymer can have more than one functional group, G. These functional groups can be chosen such that each G is modified at the same or a different temperature.

In addition, the polymer can have a group, Y, which can be photochemically or thermally cross-linked to control the softening temperature of the overall polymer, now represented by Yₙ-Pₙ-Gₙ. With the use of the Y group, the softening temperature can be tailored to be above or below the chemical modification temperature as desired. This can be accomplished by increasing or decreasing the glass transition temperature and/or the crystallinity of the polymer. The Y and G groups can be coupled to the polymer backbone through a side chain, and can be organized in blocks, which can be ordered or randomly oriented. The Y and G groups can derive from the same functional monomer unit or a different one. In some embodiments, the Y group can be chosen from cinnamate esters, chalcones, trifluorovinyl ethers, Diels-Alder reactants, or the like.

Alternatively, the surface can be formed from a self-assembled monolayer or multilayer of molecules. The molecules can be represented by the basic structure Xₙ-R-Gₙ, wherein X represents an anchoring group for the molecule to attach to a substrate or platform, R represents a bridging group, G represents the functional group that will be modified by TCNL, and n is a positive integer. These molecules can be processed by standard self-assembled monolayer- or multilayer-forming techniques, which include a reaction between a thiol-terminated Xₙ-R-Gₙ with a gold surface, silane-terminated Xₙ-R-Gₙ with a glass surface, or like reaction.

In some embodiments, the anchoring group, X, can be chosen from phosphonic acids, phosphinic acids, sulfonic acids, carboxylic acids, carbamates, dithiocarbamates, thiols, selenols, phosphines, amines, amides, carbohydroximic acids, sulfonohydroxamic acids, phosphohydroxamic acids, monochlorosilanes, dichlorosilanes, trichlorosilanes, mono(alkoxy)silanes, di(alkoxy)silanes, tri(alkoxy)silanes, or the like, or a conjugate base of any of the foregoing; the bridging group, R, can be a linear or branched C₃ to C₅₀ aliphatic or cyclic aliphatic, fluoroalkyl, oligo(ethyleneglycol), aryl, amine, or like group; and G can be any of the functional group types discussed above for polymeric surfaces.

In other embodiments, the surface can be a precursor material to a desired composition. In this manner, rather than removing a functional group or changing a functional group, a conversion from the precursor to the desired composition can occur. This can involve a reduction, an oxidation, a molecular rearrangement, or other chemical reaction, which can be tuned not only by temperature, but also by the pressure and/or atmospheric environment.

The chemically-modified surface can serve as a template for creating chemical designs thereon and/or positioning other materials thereon. These designs and/or other materials can be placed on the modified portions of the surface, or they can be placed on the unmodified portions.

The types of other materials that can be placed on the surfaces include nanoscale objects, nanoscale anchors for microscale objects, or microscale objects themselves. These materials, regardless of their size, can vary from soft and compliant materials/objects (e.g., proteins, DNA, RNA, cells, and the like) to less compliant, stiff materials (e.g., nanotubes, fullerenes, nanoparticles, and the like). There are no constraints on the nature of the other materials as their attaching force may vary as desired. Such forces for attaching the additional material/object to the surface include covalent bonding, ionic bonding, hydrogen bonding, acid-base interactions, pi-stacking, arene-perfluoroarene interactions, Van der Waals forces, methods for molecular recognition (e.g., base pairing in nucleotides, host-guest coordination receptors, and other site-specific interactions), and the like. These objects can be attached directly to the surface or through a linker and/or expander molecule that is attached to the modified surface.

By way of illustration, one application of the tSPL-patterned surface as a template involves functionalization of the tSPL-modified portions of the surface, followed by attachment of a biological material to the surface based on the biological material's affinity for a the post-tSPL functional group. This procedure is desirable in situations where the biological material ordinarily would not attach to the tSPL-modified portions of the substrate. In this manner, the additional functionalization provides the ability for the biological material to attach to the surface in the same arrangement as was patterned thereon using tSPL.

In one embodiment, the thermosensitive polymer has a stiffness that can be tuned by heat or UV light. For example, in certain embodiments, the stiffness of the polymer can be tuned with heat or UV light with value ranging from kPa to GPa. In certain embodiments, the stiffness of the thermosensitive polymer can be spatially patterned with micron scale resolution.

In certain embodiments, the fabrication of tissue replicas comprises performing tSPL on a biocompatible or cell culture compatible thermosensitive polymer, which allows for successful seeding and culturing of cells on the fabricated replicas. In one embodiment, the thermosensitive polymer comprises polymethacrylate-carbamate-cinnamate copolymer (PMCC). In one embodiment, the PMCC is adapted, and optimized from poly-((tetrahydropyran-2-yl N-(2 methacryloxyethyl) carbamate)-b-(methyl 4-(3-methacryloyloxypropoxy) cinnamate)) (see Liu et al., 2019, ACS Appl. Mater. Interfaces, 11, 41780 and Wang et al., 2009, Adv. Funct. Mater, 19, 3696, each of which are incorporated by reference in its entirety).

In one embodiment, the thermosensitive polymer (e.g, PMCC) is coated on a substrate prior to tSPL. Exemplary substrates include, but are not limited to, glass, quartz, silicon, metals, ceramics, or any other solid material. In certain embodiments, the substrate comprises a combination of materials. For example, in certain embodiments, the substrate comprises a stack of a combination of materials. In certain embodiments, the substrate is transparent which would allow for subsequent cell studies utilizing microscopy on cultured cells grown on the fabricated replicas. In one embodiment, the substrate comprises glass. In one embodiment, the substrate comprises indium tin oxide (ITO) glass. In certain embodiments, the substrate comprises a medical device, such as an orthopedic implant or dental implant.

In certain embodiments, the thermosensitive polymer (e.g., PMCC) is spin-coated on the substrate (e.g., ITO glass). An exemplary spin-coating procedure is demonstrated herein to result in optimal properties and biocompatibility, where PMCC is dissolved in chloroform (2 mg mL⁻¹) and spin coated on the ITO glasses at 1500 rotation per minute (rpm) for 60 s in a clean room environment. The coated substrates are then baked on a hot plate at 50° C. for 1 min to remove the residual solvent. This spin coating process yields uniform films with a thickness of about 10-50 nm as measured by AFM.

In one embodiment, the patterned/replicated morphology is transferred, using one or more different etching procedures, from the thermosensitive polymer to the solid substrate, such as a medical device substrate.

In certain embodiments, the method of fabricating tissue replicas comprising using an image of tissue morphology as the inputted pattern for subsequent tSPL patterning of the polymer. In certain embodiments, the image is an atomic force microscopy image, scanning electron microscopy image, and/or transmission electron microscopy image. In one embodiment, the image is an AFM amplitude error image of tissue microenvironment. For example, in certain embodiments, the image depicts an array or pattern of collagen fibrils found in the native bone microenvironment. As described herein, the gray scale of the image encodes the depth information of the resulting pattern. For example, the z-scale of the patterning can be set to match the depth information in the image. In certain embodiments, the image is repeated or arrayed to produce a larger image that can be used to fabricate larger patterned replicas.

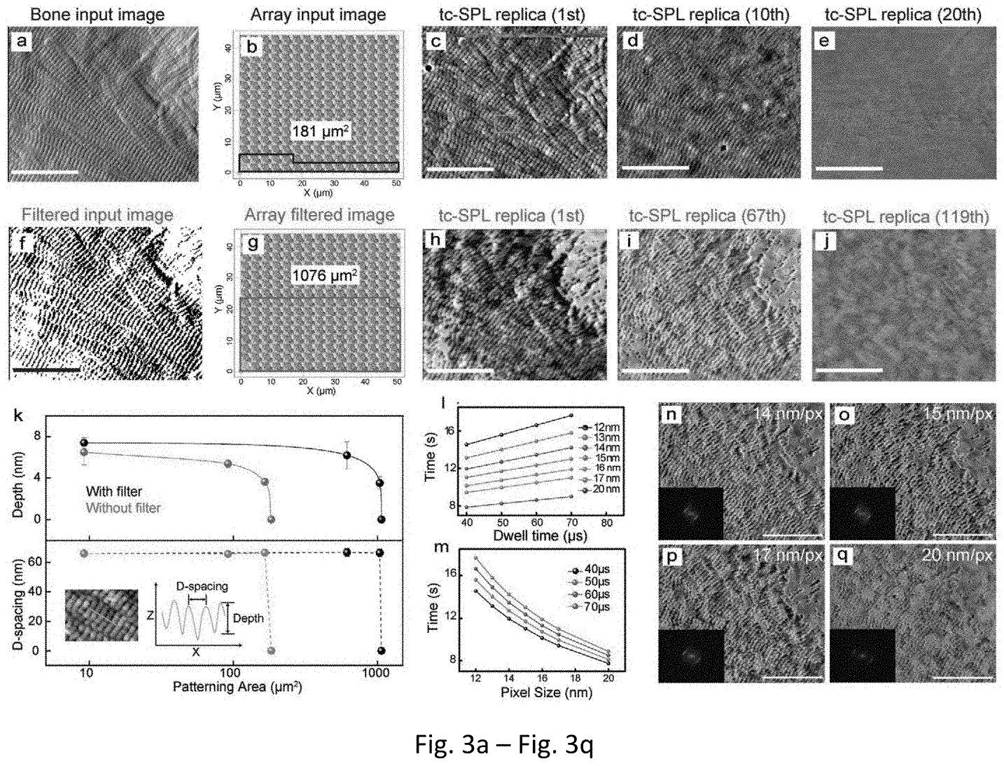

In certain embodiments, the image is processed to improve the cost and speed of patterning. For example, in one embodiment, the method comprises using a filtered image. In one embodiment, the filtering strategy comprises setting of a threshold that increases the number of 0-level pixels (above the threshold) that do not require writing. Reducing the pixels that require writing would therefore, reduce probe contamination while increasing its durability. In one embodiment, the input image is processed to separate the image into two parts, the background (higher topography) and foreground (lower topography) in order to extract useful topographical information encoded in the pixels. In one embodiment a threshold value is set to separate the image into the background and foreground: pixel values that are in modulus lower than or equal to the threshold value form the background, which are set to a 0-level and do not require writing, and pixel values greater in modulus than the threshold values form the foreground (see FIG. 3f), which is patterned by the tSPL. In one embodiment, the threshold value is equal to the mean 8-bit value of the original image, thereby separating the image into the background and foreground. However, any desired threshold value can be set that would result in filtering the image to improve the cost and speed of the patterning while retaining high resolution. In certain embodiments, the filtered or processed image is repeated or arrayed to produce a larger image that can be used to fabricate larger patterned replicas.

As discussed herein, improvements in the fabrication of biological tissue replicas were observed when optimizing certain parameters of tSPL, such as dwell time and pixel size. Optimization of such parameters allowed for improvements in patterning speed while maintaining acceptable resolution. In one embodiment, the dwell time, which corresponds to the time interval between two adjacent writing pixels, is between about 10-100 μs. In one embodiment, the dwell time is about 30-80 μs. In one embodiment, the dwell time is about 40-70 μs. In one embodiment, the dwell time is about 40-50 μs. In one embodiment, the dwell time is about 40 μs. In one embodiment, the pixel size, which corresponds to the distance between two adjacent writing pixels, is between about 10-25 nm. In one embodiment, the pixel size is about 12-20 nm. In one embodiment, the pixel size is about 14-18 nm. In one embodiment, the pixel size is about 16 nm. In one embodiment, the dwell time is about 40 μs and the pixel size is about 16 nm.

In certain embodiments, the processing and optimizing steps described herein allow for fabrication of larger tissue replicas than can be otherwise be generated. For example, the presently described methods allow for production of millimeter size replicas, which have previously unable to be produced. In one embodiment, the fabricated replicas have an area of about 1-10,000 μm². In one embodiment, the fabricated replicas have an area of about 100-8,000 μm². In one embodiment, the fabricated replicas have an area of about 200-5,000 μm². In one embodiment, the fabricated replicas have an area of about 500-2,000 μm².

It is also demonstrated herein that washing the probe periodically during use improves high-resolution writing, which reduces costs and allows for patterning of larger-scale replicas. In one embodiment, the method comprises washing the probe in a suitable solution, for example a chloroform solution. An exemplary washing procedure is described herein, where the probe was washed by dipping in a chloroform solution (98% v/v) (Sigma Aldrich) for about 1 h and dried with compressed air. However, the present invention is not limited to any particular concentration of chloroform or any particular duration of washing. In one embodiment, the probe is washed after every 1.0×10³-1.0×10⁶ μm of linear writing. In one embodiment, the probe is washed after every 1.0×10⁴-1.0×10⁵ μm of linear writing. In one embodiment, the probe is washed after every 7.0×10⁴-8.0×10⁴ μm of linear writing.

In certain embodiments, the method comprises biofunctionalizing the tissue replica. For example, in certain embodiments, tSPL, when performed at certain conditions, results in the exposure of amines on the thermosensitive polymer, where the exposed amines can then be used for conjugation or coupling of a biomolecule (e.g, a protein, growth factors, nucleic acid, carbohydrate, small molecule, or the like). In certain embodiments, the replica is biofunctionalized with biomolecules that may aid in the attachment, growth, and/or survival of seeded cells. In certain embodiments, the replica is biofunctionalized with biomolecules that are being screened to investigate whether the biomolecules influence or affect the health or function of seeded cells.

In one embodiment, the method comprises seeding cells on the fabricated tissue replica. As described herein, the present method can be used to fabricate of any biological tissue, biological compartment, or stem cell niche. Thus, any suitable cell type can be seeded on the replicas in order to generate seeded replicas that mimic native biological tissue. The cells may be from any source from any species, including a tissue bank, an autologous source, an allogenic source, or xenogeneic source. The cells may include human cells or non-human cells. Non-limiting examples of cells that can be seeded on tissue replicas include, stem cells, progenitor cells, mesenchymal stem cells, induced mesenchymal stem cells, induced pluripotent stem cells, embryonic stem cells, osteocytes, osteoblasts, osteoclasts, osteoprogenitor cells, mesenchymal cells, chondrocytes, cartilage progenitor cells, fibroblasts, endothelial cells, myocytes, cardiomyocytes, cardiac progenitor cells myoblasts, skin cells (e.g., keratinocytes, melanocytes, Langerhans cells, merkel cells), skin stem cells, tumor cells and the like. The cells may be from any source from any species, including a tissue bank, an autologous source, an allogenic source, or xenogeneic source. In certain embodiments, the cells are modified, such as genetically modified. Once seeded, the tissue replicas may be cultured using any standard media or culture conditions that support cell growth and survival.

In one aspect, the present invention provides a high resolution biological tissue replica comprising a biocompatible or cell culture compatible thermosensitive polymer coated on a substrate, where the polymer is patterned to mimic or replicate tissue microenvironment. In one embodiment, the replica comprises patterned features at a lateral resolution of less than about 15 nm. In one embodiment, the replica comprises patterned features at a vertical resolution of less than about 2 nm.

In one embodiment, the present invention provides millimeter size replicas, which have previously unable to be produced. In one embodiment, the =replicas have an area of about 1-10,000 μm². In one embodiment, the =replicas have an area of about 100-8,000 μm². In one embodiment, the replicas have an area of about 200-5,000 μm². In one embodiment, the replicas have an area of about 500-2,000 μm².

In one embodiment, the thermosensitive polymer comprises PMCC. In one embodiment, the PMCC is adapted, and optimized from poly-((tetrahydropyran-2-yl N-(2 methacryloxyethyl) carbamate)-b-(methyl 4-(3-methacryloyloxypropoxy) cinnamate)) (see Liu et al., 2019, ACS Appl. Mater. Interfaces, 11, 41780 and Wang et al., 2009, Adv. Funct. Mater, 19, 3696, each of which are incorporated by reference in its entirety).

In one embodiment, the thermosensitive polymer (e.g, PMCC) is coated on the substrate. Exemplary substrates include, but are not limited to, glass, quartz, silicon, metals, ceramics, or any other solid material. In certain embodiments, the substrate comprises a combination of materials. For example, in certain embodiments, the substrate comprises a stack of a combination of materials. In certain embodiments, the substrate is transparent which would allow for subsequent cell studies utilizing microscopy on cultured cells grown on the fabricated replicas. In one embodiment, the substrate comprises glass. In one embodiment, the substrate comprises indium tin oxide (ITO) glass. In certain embodiments, the substrate comprises a medical device, such as an orthopedic implant or dental implant.

In certain embodiments, the thermosensitive polymer (e.g., PMCC) is spin-coated on the substrate (e.g., ITO glass).

In certain embodiments, the present invention provides tissue replicas fabricated using localized heat, such as tSPL, as described herein. For example, as described herein, the replicas fabricated using the optimized tSPL methodologies described herein are of larger size and high resolution, and are able to be fabricated at low cost and at high throughput.

In one embodiment, the replicas further comprise one or more cell types seeded on the replicas. Any suitable cell type can be seeded on the replicas in order to generate seeded replicas that mimic native biological tissue. The cells may be from any source from any species, including a tissue bank, an autologous source, an allogenic source, or xenogeneic source. The cells may include human cells or non-human cells. Non-limiting examples of cells that can be seeded on tissue replicas include, stem cells, progenitor cells, mesenchymal stem cells, induced mesenchymal stem cells, induced pluripotent stem cells, embryonic stem cells, osteocytes, osteoblasts, osteoclasts, osteoprogenitor cells, mesenchymal cells, chondrocytes, cartilage progenitor cells, fibroblasts, endothelial cells, myocytes, cardiomyocytes, cardiac progenitor cells myoblasts, skin cells (e.g., keratinocytes, melanocytes, Langerhans cells, merkel cells), skin stem cells, tumor cells and the like. The cells may be from any source from any species, including a tissue bank, an autologous source, an allogenic source, or xenogeneic source. In certain embodiments, the cells are modified, such as genetically modified.

In some embodiment, the present invention provides methods of using the tissue replicas described herein. For example, the tissue replicas can be used for a variety of cellular experiments or assays. Cells seeded and cultured on the replicas can be stained with dyes or fluorescent molecules, or used in immunocytochemistry or immunohistochemistry to visualize the cells, assess the health of the cells, and/or to detect the presence of a biomolecule of interest.

The cells, cellular components (e.g., proteins, DNA, and RNA), or media obtained from the replicas can be analyzed using any methodology known in the art. For example, cells can be stained and/or analyzed using immunofluorescence, immunocytochemistry, immunohistochemistry, or the like. In certain embodiments, the cells may be lysed to analyze protein expression, RNA expression, etc. Exemplary techniques used to analyze the cells or media obtained from the device includes, but is not limited to, DNA sequencing, RNA sequencing, PCR, RT-PCR, protein sequencing, immunoblotting, immunoprecipitation, ELISA, mass spectrometry, crystallography, and the like. Further, cells obtained from the replicas can further be subjected to one or more cellular assays to evaluate the function of the obtained cells. As a skilled artisan would readily understand, the present invention is not limited to any particular analysis, technique, or assay; but rather any suitable analysis, technique, or assay may be conducted on cells, media, or cellular components (e.g., proteins, DNA, and RNA) obtained from the replicas.

In some embodiments, the present invention provides methods for screening for agents that may affect or influence the function of cells (e.g., bone cells, osteoblasts, osteoclasts, progenitor cells, stem cells) seeded on the described tissue replicas. In some embodiments, the present invention can be used to screen for agents with potential therapeutic effects. In certain embodiments, the method comprises screening for agents that may affect or influence cell motility, migration, differentiation, proliferation, or the like. The method may also comprise screening one or more agents for the ability to increase or decrease the expression of a marker, such as a protein or nucleic acid marker. The present invention provides a more realistic biomimetic platform that allows for more faithful screening assays. In certain embodiments, the screening methods described herein are performed on a subject's own cells thereby providing a personalized screening method to identify agents that may work specifically for the subject.

Exemplary agents that can be screened include, not are not limited to, a small interfering RNA (siRNA), a sguide RNA (gRNA), a microRNA, an antisense or sense nucleic acid, a ribozyme, a nucleic acid molecule, an antibody, a peptide, a chemical compound and a small molecule.

As demonstrated herein, the replicas described herein are reusable. Thus, in certain embodiments, cells and media can be removed from the replicas, the replicas can be washed, and re-seeded with a different population of cells for further experimentation.

In certain embodiments, the tissue replicas can be transplanted into a subject in need thereof. For example, as described elsewhere herein, in certain embodiments, the replicas comprise a medical device substrate, such as an orthopedic implant or dental implant, wherein the substrate and/or polymer is patterned to replicate biological tissue morphology. In certain embodiments, the medical device substrate can be implanted or transplanted into a subject to support growth of cells or tissue on the substrate.

In one aspect, the present invention provides a system for generating a tissue replica. For example, in one embodiment, the system comprises a tSPL device, a substrate and a thermosensitive polymer. As described elsewhere herein, in one embodiment the tSPL device comprises a probe (such as an AFM tip or AFM instrument) and a resistive heater in electrical communication with the probe. In one embodiment, the substrate is composed of glass, quartz or silicon. In one embodiment, the substrate is transparent. In one embodiment, the substrate comprises ITO glass. In one embodiment, the thermosensitive polymer is biocompatible or cell culture compatible. For example, in one embodiment, the thermosensitive polymer comprises PMCC. In one embodiment, the system comprises the thermosensitive polymer (e.g., PMCC) coated (e.g., spin-coated) on the substrate (e.g., ITO glass).

In one embodiment, the system comprises a computing device, such as a computer, laptop, tablet, or the like. In certain embodiments, the computing device receives an inputted image (such as an AFM, SEM, or TEM image) of a tissue or tissue microenvironment and communicates with the tSPL device to pattern to polymer-coated substrate with a pattern that mimics the topography of the inputted image. In certain embodiments, the computing device performs one or more processing steps on the imputed image. For example, in certain embodiments, the computing device creates an array of the inputted image to generate a larger pattern.

In certain embodiments, the system further comprises at least one cell type for seeding and culturing on the tissue replica.

In some aspects of the present invention, software executing the instructions provided herein may be stored on a non-transitory computer-readable medium, wherein the software performs some or all of the steps of the present invention when executed on a processor.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled or interpreted, including but not limited to C, C++, C#, Objective-C, Java, JavaScript, MATLAB, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G, 4G/LTE, or 5G networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

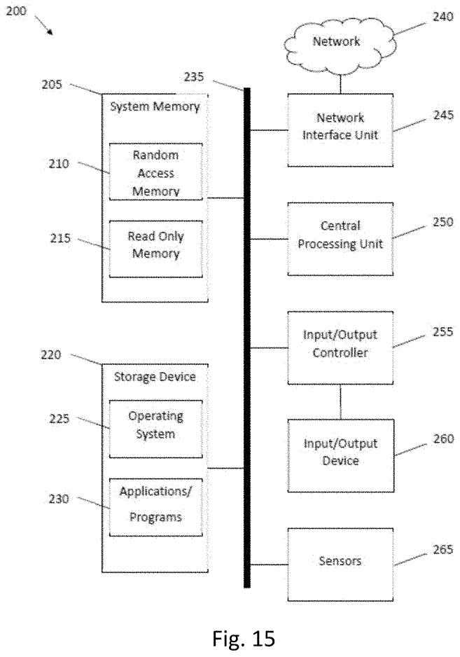

FIG. 15 and the following discussion are intended to provide a brief, general description of a suitable computing environment in which the invention may be implemented. While the invention is described above in the general context of program modules that execute in conjunction with an application program that runs on an operating system on a computer, those skilled in the art will recognize that the invention may also be implemented in combination with other program modules.

Generally, program modules include routines, programs, components, data structures, and other types of structures that perform particular tasks or implement particular abstract data types. Moreover, those skilled in the art will appreciate that the invention may be practiced with other computer system configurations, including hand-held devices, multiprocessor systems, microprocessor-based or programmable consumer electronics, minicomputers, mainframe computers, and the like. The invention may also be practiced in distributed computing environments where tasks are performed by remote processing devices that are linked through a communications network. In a distributed computing environment, program modules may be located in both local and remote memory storage devices.

FIG. 15 depicts an illustrative computer architecture for a computer 200 for practicing the various embodiments of the invention. The computer architecture shown in FIG. 15 illustrates a conventional personal computer, including a central processing unit 250 (“CPU”), a system memory 205, including a random access memory 210 (“RAM”) and a read-only memory (“ROM”) 215, and a system bus 235 that couples the system memory 205 to the CPU 250. A basic input/output system containing the basic routines that help to transfer information between elements within the computer, such as during startup, is stored in the ROM 215. The computer 200 further includes a storage device 220 for storing an operating system 225, application/program 230, and data.

The storage device 220 is connected to the CPU 250 through a storage controller (not shown) connected to the bus 235. The storage device 220 and its associated computer-readable media provide non-volatile storage for the computer 200. Although the description of computer-readable media contained herein refers to a storage device, such as a hard disk or CD-ROM drive, it should be appreciated by those skilled in the art that computer-readable media can be any available media that can be accessed by the computer 200.

By way of example, and not to be limiting, computer-readable media may comprise computer storage media. Computer storage media includes volatile and non-volatile, removable and non-removable media implemented in any method or technology for storage of information such as computer-readable instructions, data structures, program modules or other data. Computer storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

According to various embodiments of the invention, the computer 200 may operate in a networked environment using logical connections to remote computers through a network 240, such as TCP/IP network such as the Internet or an intranet. The computer 200 may connect to the network 240 through a network interface unit 245 connected to the bus 235. It should be appreciated that the network interface unit 245 may also be utilized to connect to other types of networks and remote computer systems.

The computer 200 may also include an input/output controller 255 for receiving and processing input from a number of input/output devices 260, including a keyboard, a mouse, a touchscreen, a camera, a microphone, a controller, a joystick, or other type of input device.

Similarly, the input/output controller 255 may provide output to a display screen, a printer, a speaker, or other type of output device. The computer 200 can connect to the input/output device 260 via a wired connection including, but not limited to, fiber optic, Ethernet, or copper wire or wireless means including, but not limited to, Wi-Fi, Bluetooth, Near-Field Communication (NFC), infrared, or other suitable wired or wireless connections.

As mentioned briefly above, a number of program modules and data files may be stored in the storage device 220 and/or RAM 210 of the computer 200, including an operating system 225 suitable for controlling the operation of a networked computer. The storage device 220 and RAM 210 may also store one or more applications/programs 230. In particular, the storage device 220 and RAM 210 may store an application/program 230 for providing a variety of functionalities to a user. For instance, the application/program 230 may comprise many types of programs such as a word processing application, a spreadsheet application, a desktop publishing application, a database application, a gaming application, internet browsing application, electronic mail application, messaging application, and the like.

According to an embodiment of the present invention, the application/program 230 comprises a multiple functionality software application for providing word processing functionality, slide presentation functionality, spreadsheet functionality, database functionality and the like.

The computer 200 in some embodiments can include a variety of sensors 265 for monitoring the environment surrounding and the environment internal to the computer 200. These sensors 265 can include a Global Positioning System (GPS) sensor, a photosensitive sensor, a gyroscope, a magnetometer, thermometer, a proximity sensor, an accelerometer, a microphone, biometric sensor, barometer, humidity sensor, radiation sensor, or any other suitable sensor.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Cost and Time Effective Lithography of Reusable Millimeter Size Bone Tissue Replicas with Sub-15 nm Feature Size on a Biocompatible Polymer

Here, a novel platform is reported that combines thermal scanning probe lithography (tSPL) with innovative methodologies for the low-cost and high-throughput nanofabrication of large area quasi-3D bone tissue replicas with high fidelity, sub-15 nm lateral precision, and sub-2 nm vertical resolution. This bio-tSPL platform features a biocompatible polymer resist that withstands multiple cell culture cycles, allowing the reuse of the replicas, further decreasing costs and fabrication times. The as-fabricated replicas support the culture and proliferation of human induced mesenchymal stem cells, which display broad therapeutic and biomedical potential. Furthermore, it is demonstrated that bio-tSPL can be used to nanopattern the bone tissue replicas with amine groups, for subsequent tissue-mimetic biofunctionalization. The achieved level of time and cost-effectiveness, as well as the cell compatibility of the replicas, make bio-tSPL a promising platform for the production of tissue-mimetic replicas to study stem cell-tissue microenvironment interactions, test drugs, and ultimately harness the regenerative capacity of stem cells and tissues for biomedical applications.

Described herein is a new bio-tSPL platform that dramatically reduces the problems of throughput and high cost of tSPL by introducing smart software and post-patterning procedures, as well as a biocompatible, functional polymer resist that it is shown to withstand multiple cell culture cycles, allowing the reuse of the tissue replicas. Using this platform, we replicate the complex quasi-3D morphology of the bone tissue over millimeter scale areas, with sub-15 nm resolution in x-y, and sub-2 nm resolution in z-direction. The millimeter replicas are fabricated with increased throughput and reduced cost such that their production becomes now feasible for biomedical research. To enable cell studies, the polymer resist is spin-coated on a transparent indium tin oxide (ITO) glass, and its cell culture compatibility is demonstrated to be as good as conventional tissue culture plastic materials. Furthermore, we show that is possible to chemically functionalize with nanoscale spatial resolution the bone tissue topographical replicas with ad hoc molecules (X. Y. Liu, et al., ACS Appl. Mater. Interfaces 2019, 11, 41780; D. B. Wang, et al., Adv. Funct. Mater. 2009, 19, 3696; E. Albisetti, et al., Nanotechnology 2016, 27, 315302; K. M. Carroll, et al., Langmuir 2013, 29, 8675). Finally, we demonstrate that the as-fabricated bone replicas on the biocompatible polymer surface support adhesion and proliferation of mesenchymal cells derived from human induced pluripotent stem cells (iMSCs) (G. M. de Peppo, et al., Proc. Natl. Acad. Sci. USA 2013, 110, 8680), which can be derived in large numbers from each individual, and can differentiate into multiple cell types (K. Takahashi, et al., Cell 2007, 131, 861) for personalized biomedical applications.

The methods used in these experiments are now described.

Substrate and Film Preparation: ITO-coated glasses (1 cm×1 cm) with a thickness of 1.1 mm (MSE Supplies, LLC, Tucson, Ariz.) were cleaned by ultrasonication in acetone, methanol, and isopropanol alcohol for 3 min. To increase adhesion and remove organic residuals 02 plasma treatments are used. Then, the PMCC resist, adapted, and optimized from poly-((tetrahydropyran-2-yl N-(2 methacryloxyethyl) carbamate)-b-(methyl 4-(3-methacryloyloxypropoxy) cinnamate)) (X. Y. Liu, et al., ACS Appl. Mater. Interfaces 2019, 11, 41780; D. B. Wang, et al., Adv. Funct. Mater. 2009, 19, 3696), was dissolved in chloroform (2 mg mL⁻¹) and spin coated on the ITO glasses at 1500 rotation per minute (rpm) for 60 s in a clean room environment. The samples were then baked on a hot plate at 50° C. for 1 min to remove the residual solvent. This spin coating process yields uniform films with a thickness of about 10-50 nm as measured by AFM. The film preparation conditions and properties (e.g., adhesion to the substrate, thickness, and surface roughness) have been optimized to achieve (i) optimal resist-substrate adhesion for prolonged stability (at least one month) in a hydrolytic cell culture environment, without film delamination from the substrate, (ii) biocompatibility and excellent cell adhesion without the need of any additional chemical treatment of the surface; and (iii) reusability of the patterned films for multiple cell culture cycles (here it has been demonstrated up to 7 cycles), which considerably increases the number of cell biology tests that can be performed with one replica. Very importantly, the excellent resist-substrate adhesion of the film to the substrate and the robustness of the pattern on the resist is further demonstrated by their ability to withstand not only the cell culture but also the treatment with trypsin that is necessary for cell detachment and removal, and reuse of the replica (see FIG. 6).

Thermal-SPL: Patterning of the polymer resist was performed using a commercial tSPL system (NanoFrazor, Heidelberg Instruments, Germany), which utilizes a heated silicon probe for patterning the PMCC resist. For patterning, the probe on the head of the thermal cantilever is heated up by a resistive micron-heater. The probe works as a separate thermal reading sensor for topography thermal imaging when the microheater is turned off. During the patterning, the thermal reading sensor probed the topography of the patterned structure right after each patterning line when retracing back in contact mode, which leads to the simultaneous patterning and imaging capability of the NanoFrazor system as well as a closed feedback loop correction. The probe temperature is automatically calibrated through the system software according to the current-voltage characteristics of the Si tip and its theoretical value of knee point (U. Durig, J. Appl. Phys. 2005, 98, 044906). In this study, the probe temperatures used were mostly around 800-900° C. For patterning different amine densities on the bone replicas, different probe temperatures, i.e., 875, 850, 825, and 800° C. were used for each row from top to bottom, while all other patterning parameters were kept constant.

Image Threshold Filtering: The filtering strategy involves the setting of a threshold that increases the number of 0-level pixels (above the threshold) that do not require writing; therefore, reducing probe contamination while increasing its durability. The AFM bone input image was processed to separate the image into two parts, the background (higher topography) and foreground (lower topography) in order to extract useful topographical information encoded in the pixels. Briefly, a threshold value equal to the mean 8-bit value of the original image was set and the original image was divided into two portions: pixel values that are in modulus lower than or equal to the threshold value form the background, which are set to a 0-level and do not require writing, and pixel values greater in modulus than the threshold values form the foreground (see FIG. 3f), which is patterned by the tSPL.

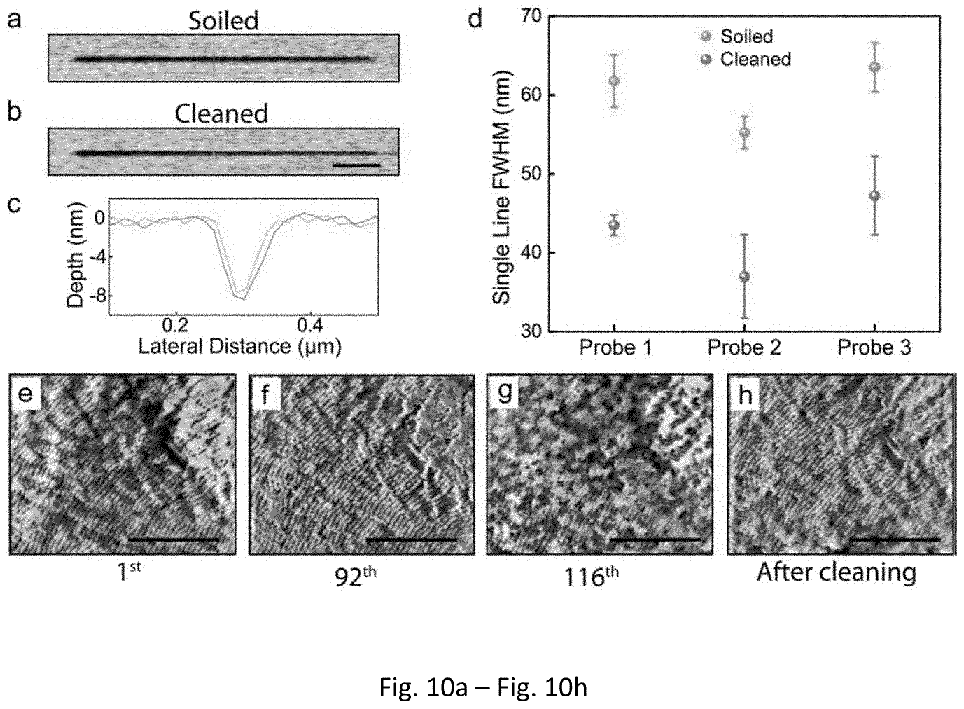

Thermal Probe Cleaning: The probe was washed by dipping in a chloroform solution (98% v/v) (Sigma Aldrich) for 1 h and dried with compressed air. Chloroform was chosen as it is the solvent for the polymer solution. Results for the thermal probe cleaning tests are described elsewhere herein and in FIG. 10. This cleaning protocol was employed for the fabrication of large bone replica.

Atomic Force Microscopy Characterization: All AFM experiments were performed on a Bruker MultiMode 8 AFM using tapping mode. Flattening and z-scale adjustment of AFM images were performed with the Gwyddion software.

Cell Culture: Human iMSCs (line 1013A) were derived and characterized as previously described (G. M. de Peppo, et al., Proc. Natl. Acad. Sci. USA 2013, 110, 8680). For seeding, cells were thawed, centrifuged at 1000 rpm for 5 min, and resuspended in expansion medium consisting of high-glucose KnockOut Dulbecco's modified Eagle's medium (DMEM; Gibco) supplemented with 20% v/v HyClone fetal bovine serum (FBS; GE Healthcare Life Sciences), beta-fibroblast growth factor (1 ng mL⁻¹; Invitrogen), nonessential amino acids (0.1×10⁻³ m; Gibco), GlutaMAX (2×10⁻³ m; Gibco), betamercaptoethanol (0.1×10⁻³ m; Gibco), and antibiotic-antimycotic (100 U mL⁻¹; Gibco). Aliquots of cell suspension were then added to the PMCC-coated ITO glasses placed in ultra-low attachment plates (Fisher Scientific) at a density of ≈1000-10 000 cells cm⁻². Following seeding, samples were placed in a humidified environment at 37° C. for 3 h to let cells attach, and then cultured in expansion medium for up to 7 d. Cell seeding, attachment, and growth were monitored using the EVOS FL Cell Imaging System (Life Technologies). To test the reusability of the PMCC-coated ITO glasses, cells were seeded as described above and samples cultured in osteogenic medium consisting of high-glucose DMEM medium supplemented with 10% v/v HyClone FBS (GE Healthcare Life Sciences), dexamethasone (1×10⁻⁶ m; Sigma), beta-glycerophosphate (10×10⁻⁶ m; Sigma), ascorbic acid-2-phosphate (50×10⁻⁶ m; Sigma), and antibiotic-antimycotic (100 U mL⁻¹, Gibco) for 3 d. At the end of each culture experiment, cells were detached by incubation with trypsin (0.25% Trypsin EDTA; Fisher Scientific) for 10 min. Samples were then thoroughly washed in phosphate buffer saline (PBS) solution and distilled H₂O for 5 min, airdried using a dust remover, and stored in ambient conditions at room temperature until reuse.

PrestoBlue Assay: The metabolic activity of the cells was estimated using the PrestoBlue reagent (Life Technologies), a cell permeable resazurin-based solution that functions as an indicator of the cell metabolic activity by using the reducing power of living cells. Briefly, samples were treated with 1 mL of expansion medium containing 10% v/v of PrestoBlue reagent (Life Technologies), and incubated for 1 h at 37° C. Following incubation, 200 μL aliquots of culture media were transferred to a black, clear, flat-bottom 96-well plate (BD Falcon™), and fluorescence measured at 560/590 nm (excitation/emission) using the fluorescent reader SYNERGYMx (BioTek) equipped with Gen 5 1.09 software.

Viability Assays: The biocompatibility of the PMCC polymer was assessed using the LIVE/DEAD (Thermo Scientific) and the acridine orange (AO) and propidium iodide (PI) viability assay (Invitrogen). Briefly, 1000 cells were seeded on the PMCC polymer and cultured in expansion medium for 7 d. For LIVE/DEAD assay, samples were washed with PBS and incubated with a solution of calcein AM (2×10⁻³ m) and ethidium homodimer-1 (4×10⁻³ m) in PBS at 37° C. in the dark for 10 min. Following incubation, samples were washed in PBS (Gibco), and then placed in RPMI medium (without red phenol; Lonza) for imaging. Confocal images were taken with the Axiovert 200M microscope (Carl Zeiss AG) mounted with LSM 5 Pascal exciter and using the LSM 5 Pascal software with defined settings. Cells treated with distilled H₂O for 20 min were used as positive control for the ethidium homodimer-1 staining. Quantification of live (green) and dead (red) cells was conducted in ImageJ (National Institutes of Health) using the open source image processing package Fiji. Briefly, original confocal images were split into two channels, and the area covered by live and dead cells was measured with pixel values set to 40 and 80, respectively. Data are shown as the percentage area of live cells per area of total cells. For the AO/PI viability assay, cells were detached using trypsin/EDTA (0.25%, Thermo Fisher Scientific) and then analyzed using the Cellometer K2 Fluorescent Viability Cell Counter (Nexcelom Bioscience). Cells seeded on traditional tissue culture plastic were used as reference for all experiments.

Cell Painting: Cell morphology and the presence of focal contacts with the substrate were assessed using the CHEMICON's Actin Cytoskeleton and Focal Adhesion Staining Kit (Millipore Sigma) according to the manufacturer's instructions. Briefly, cells were thawed and seeded onto the polymer-coated ITO glasses at a density of ≈10 000 cells cm⁻². Following incubation in expansion medium at 37° C. overnight, cells were fixed using a 4% v/v paraformaldehyde solution in PBS (Chem Cruz, Santa Cruz) for 15 min. Thereafter, samples were permeabilized with 0.1% v/v Triton in PBS, washed twice in PBS, and stained with TRITC-conjugated phalloidin and an antivinculin mouse monoclonal antibody. A goat antimouse Alexa Fluor secondary antibody (Thermofisher Scientific; cat #A-21042) was used for vinculin detection. Nuclei were counterstaining with DAPI. Samples were stored in PBS a 4° C. before imaging.

Chemical Functionalization of Bone Tissue Replica: Alexa 488 dyes were immobilized onto the bone tissue replica patterns by incubating the polymer film with 100 μL of Alexa 488 solution (100×10⁻³ m) in H₂O for 45 min. After incubation, the surface was rinsed with distilled H₂O and then dried with Na.

Fluorescence Microscopy Imaging: Fluorescence of the bone tissue topography pattern was measured using laser scanning confocal microscopy (Zeiss LSM 880 Airyscan). The fluorescence signal was collected with excitation laser wavelength λ=488 nm and emission detection range λ=510-600 nm. The optical contrast in different fluorescent images was tuned for better visibility by standard image processing using ImageJ. Confocal images of cells growing on the bone tissue replica were taken with the microscope Axiovert 200M microscope (Zeiss, Oberkochen, GE) mounted with LSM 5 Pascal exciter using the LSM 5 Pascal software (Zeiss) using defined settings.

Scanning Electron Microscopy: SEM imaging was used to examine the morphological characteristics of the cells attached to PMCC spin coated on ITO glass. One day after cell seeding, the samples were washed with PBS and then fixed with a 2.5% v/v solution of glutaraldehyde in sodium cacodylate buffer (0.1 m, Electron Microscopy Sciences) at room temperature for 15 min. Samples were finally rinsed in distilled H₂O, placed onto SEM stubs using carbon tape, sputtered with a 5 nm layer of Pt, and imaged with a Zeiss Merlin (Zeiss) equipped with a high efficiency secondary electron detector and operated at an acceleration voltage of 3 kV and a probe current of 100 pA.

The results of these experiments are now described.

tSPL Process Scheme for Producing Large-Scale Bone Tissue Replicas

tSPL can write topographical and chemical features in a thermosensitive polymer resist by local heating, thereby carving and chemically activating the surface with nanoscale precision (X. Y. Liu, et al., ACS Appl. Mater. Interfaces 2019, 11, 41780; D. B. Wang, et al., Adv. Funct. Mater. 2009, 19, 3696). The tSPL process involves first the pixilation of a reference input image, and then the replication of that image by assigning at each grey level of individual pixels a particular height level. For biomedical oriented applications, the main drawback of SPL techniques, including tSPL, is the limited throughput due to the serial writing process, the low writing speed, and the high cost associated with large scale patterning. Furthermore, it is of key importance to identify a substrate that is transparent, cell-compatible, and stable in a wet hydrolytic environment at 37° C.

Here, we introduce a series of steps to enable the replication of the bone tissue morphology over large areas with nanometer resolution, i.e., sub-2 nm in z-direction and 15 nm in x-y direction, on a stable, cell culture compatible polymer resist spin-coated on a transparent ITO glass. In FIG. 1a-FIG. 1i we present a diagram showing all the key steps of the bio-tSPL process. First, we upload an atomic force microscopy (AFM) image bitmap input image of the bone microenvironment in the tSPL software (FIG. 1a,b). We then apply a series of input image filtering processes, and patterning parameters optimization strategies to increase throughput and decrease cost. Using these procedures and parameters, we then pattern large scale bone tissue replicas in the cell culture compatible polymethacrylatecarbamate-cinnamate copolymer (PMCC) resist (X. Y. Liu, et al., ACS Appl. Mater. Interfaces 2019, 11, 41780; D. B. Wang, et al., Adv. Funct. Mater. 2009, 19, 3696), adapted and optimized here for biocompatibility and cell culture studies, see the Experimental Section for more details (FIG. 1d). The main source of the operational costs in tSPL is the large number of thermal probes required for patterning, due to the probe contamination occurring after patterning large areas. Therefore, here we demonstrate the possibility to clean the probes by washing them in an ad hoc solution (FIG. 1e,f), see the Experimental Section. Once the replicas are ready, we culture human iMSCs on them (FIG. 1g). Finally, to further decrease costs and timing, we show that the replicas are reusable for multiple cell studies, after removing the cells and cleaning the sample following each cell culture experiment (FIGS. 1h-j and 6).

### Cell Culture Compatibility of the PMCC Resist

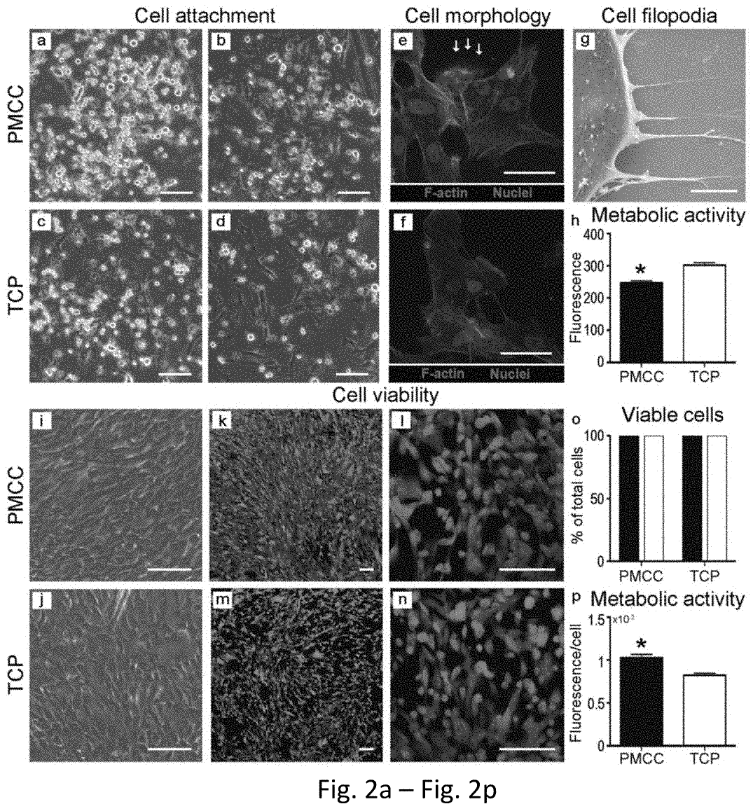

Typically, tSPL is performed on Si substrates. However, a transparent substrate is required for cell studies to control cell seeding and monitor cell adhesion, morphology, and proliferation during culture. Previous applications of tSPL for cell studies have used an adhesion layer to stabilize the binding of the PPA resist to the not transparent Si substrate (S. W. Tang, et al., ACS Appl. Mater. Interfaces 2019, 11, 18988). Here, we spin coat a thin film of the thermosensitive PMCC resist directly on a ITO glass substrate, and study the ability of this new system to support the growth of iMSCs, as well as to withstand the wet hydrolytic environment at 37° C. iMSCs can be derived in large numbers from each individual, and can differentiate into multiple cell types (K. Takahashi, et al., Cell 2007, 131, 861), thus representing an ideal cell source for personalized biomedical applications. The results show that PMCC supports the culture of human iMSCs similarly to standard polystyrene tissue culture plastic (TCP) (FIG. 2). Cells quickly attach to both substrates, although slightly more efficiently on TCP than on PMCC, as evidenced by light microscopy examination (FIG. 2a-d), and display equivalent size and typical spindle-like morphology 1 d after seeding, as shown by fluorescence staining of F-actin cytoskeletal filaments (FIG. 2e,f). Closer examination via scanning electron microscopy (SEM) imaging demonstrates that the cells extend slender cytoplasmic projections—filopodia—as connection points to the PMCC (FIG. 2g), which are important for cell adhesion and migration (P. K. Mattila, et al., Nat. Rev. Mol. Cell Biol. 2008, 9, 446). Furthermore, calcein/ethidium homodimer 1 and acridine orange/propidium iodide stainings after 7 d of cell culture (FIG. 2i-n) show that the PMCC polymer supports cell culture equally well as traditional TCP,

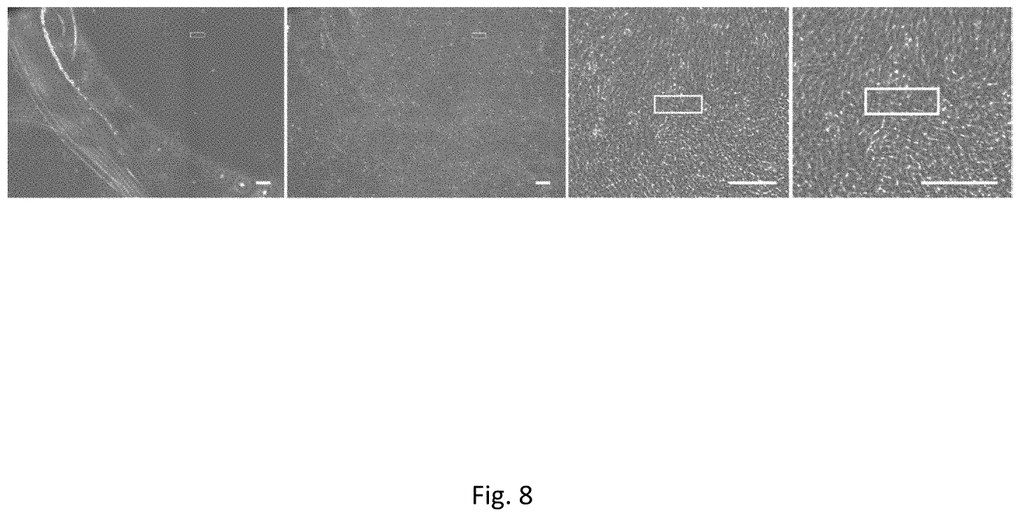

with both substrates displaying more than 99% cell viability. In addition, a PrestoBlue assay reveals that cells display higher metabolic activity for cells cultured on PMCC than on TPC (see FIG. 2p), excluding any toxic effect of the PMCC resist (or the patterned replicas, see FIG. 8) on human iMSCs. In summary, these results demonstrate the suitability of this new platform to culture cells and study the effects of the tissue microenvironment replicas on cell behavior.

### Increasing Throughput and Decreasing Cost of Replicas for Cell Culture Studies

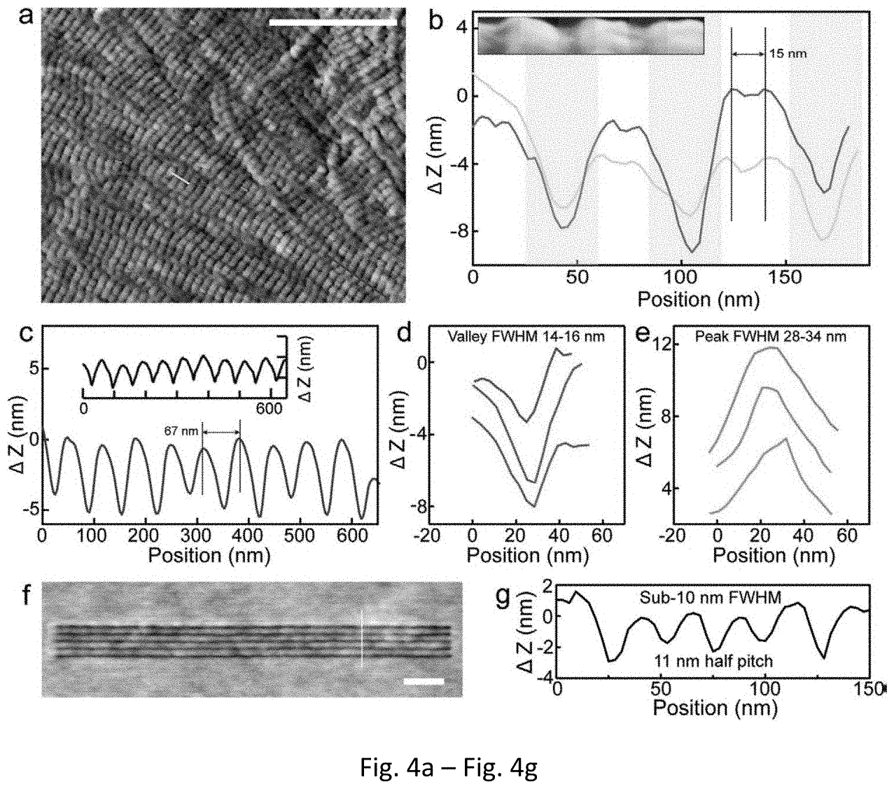

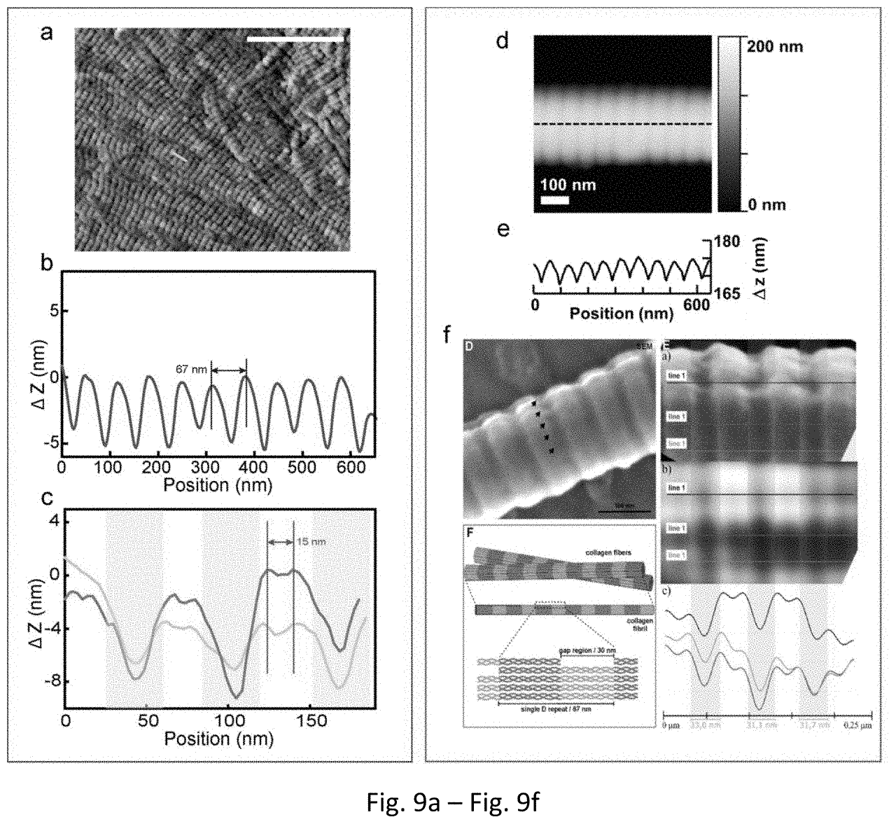

To reproduce the bone tissue topography in the PMCC resist, we utilize high-resolution images of native collagen fibrils (FIGS. 3 and 4). We first use an amplitude error AFM image of a demineralized bone tissue section (J. M. Wallace, et al., Langmuir 2010, 26, 7349) as a tSPL bitmap input, in which the depth information is encoded by gray scale (FIG. 3a). Then, since during the fabrication process we can set the z-scale of the patterning as desired, we use a high-resolution AFM height image of a single type I collagen fibril (see FIG. 4c and FIG. 9a) and set the patterning z-scale in order to replicate the dimension of this collagen fibril. We then reproduce an array of these bone input AFM images to scale up the size of the replica. In particular, in FIG. 3b we show a 50 μm×50 μm array. FIG. 3c shows the thermal image, acquired in-situ during patterning (X. R. Zheng, et al., Nat. Electron. 2019, 2, 17) of the first replicated bone input image in the array (see the Experimental Section for more details). The quality of this replica confirms the capability of tSPL to pattern complex quasi-3D tissue microenvironments with high fidelity and resolution (see more details about resolution in FIG. 4). Due to probe contamination occurring during patterning, the quality and resolution of the replica decrease quite dramatically, eventually hindering further patterning completely. This effect is clearly observed in FIG. 3c-e, being respectively the first, 10th, and 20th reproduction of the bone input image shown in FIG. 3a. These results indicate that to maintain the required spatial resolution, only 181 μm² can be patterned with a single probe without significantly compromising the quality and resolution of the bone tissue replica. As a consequence, to pattern 0.5 mm×0.5 mm size replicas, it would require changing the thermal probe 1381 times. Here, we demonstrate that by adopting a threshold filtering strategy of the input image (FIG. 3f,g), we can pattern uninterruptedly 1076 μm² replicas with acceptable quality and resolution using a single probe. This strategy reduces the number of times required to change the thermal probe by 500%, a factor 6 compared to the unfiltered case, i.e., 232 times to pattern 0.5 mm×0.5 mm size replicas. This reduction has a major impact in decreasing the costs and increasing the throughput since changing the probe requires time. This filtering strategy involves the setting of a threshold (FIG. 3f), which increases the number of 0-level pixels (above the threshold) that do not require writing (and hence probe-surface contact); therefore, reducing probe contamination while increasing its durability. FIG. 3k shows how the patterning of the bone tissue replicas maintains a good spatial resolution for larger patterning areas when the input image is filtered (FIG. 3f) compared to the unfiltered bone image (FIG. 3a). In particular, in FIG. 3k we plot, as a function of patterning area, the depth and D-spacing periodicity of the nanostructures typical of the collagen fibrils in the ECM of the bone tissue (J. M. Wallace, et al., Langmuir 2010, 26, 7349; J. M. Wallace, et al., Bone 2010, 46, 1349), as shown in FIG. 3a. The results show that within an area of 181 μm², for the original bone input image, and 1076 μm² for the filtered input image, the depth (4-8 nm) and the periodic D-spacing (67 nm) of the replicated collagen fibrils are within the typical values reported in literature for the bone tissue microenvironment (J. M. Wallace, et al., Langmuir 2010, 26, 7349; J. A. Petruska, et al., Proc. Natl. Acad. Sci. USA 1964, 51, 871; H. N. Su, et al., Nanoscale 2014, 6, 8134). The collagen fibrils have a complex structure as shown in FIGS. 9d and 9f. In particular, tropocollagen molecules assemble in a specific arrangement resulting in the formation of fibrils with gaps of about 20-30 nm, and bumps of about 40 nm, as replicated and shown in our replica (FIG. 4). The here replicated collagen fibrils also show the characteristic twin-peak structure of the fibril bumps (J. F. W. Greiner, et al., Nanomed. Nanotechnol. 2019, 17, 319), having a distance between them of ≈15-20 nm (FIG. 4b). Importantly, we replicate features that are equal or larger than 15 nm (FIG. 4b-e) because this is the size of the molecular structures that control the interaction of the cells with the ECM in the human body (P. C. Paul, et al., Nanotechnology 2011, 22, 275306). FIG. 4f,g shows the resolution of tSPL for patterning structures in PMCC.

To further reduce the costs related to the large number of thermal probes needed for large-scale replicas, we introduce a probe washing procedure based on cleaning the probe in chloroform after every 7.7×10⁴ μm of linear writing in the case of the filtered input image. FIG. 10 demonstrates the ability to clean the tip and to recover high-resolution writing, which reduces costs and creates new possibilities for large-scale patterning.

Three soiled probes were used to test the cleaning effect. A single line was first patterned before the cleaning (FIG. 10a) and then its cross-sectional profile and full width at half maximum (FWHM) were compared with a single line patterned after the cleaning (FIGS. 10b and 10c) by using the same probe (probe #1). The same experiment was performed for all the three probes, and the FWHM of the patterned lines before and after cleaning are shown in FIG. 10d, which demonstrates the restored high resolution as indicated by a reduction in FWHM of 15-20 nm after cleaning with chloroform. This cleaning protocol was employed for the fabrication of large bone replica. Several small squared bone replicas were fabricated and imaged sequentially (FIG. 10e-10h). After 1050 μm² the thermal probe was washed as described above. The small squared bone replica in FIG. 10h shows that the resolution and quality of the bone tissue replica is restored after washing the probe, demonstrating the efficacy of the proposed cleaning method.

Furthermore, to improve the patterning speed while maintaining a faithful bone topographical reproduction, we optimize the pixel dwell-time, t_{dwell} (40-70 μs) and pixel size, dₚᵢₓₑₗ (12-20 nm). Here, the pixel dwell-time and pixel size correspond to the time interval and the distance between two adjacent writing pixels, respectively. The time needed to pattern each filtered input image (FIG. 3f) as a function of both pixel dwell-time and pixel size is plotted in FIG. 3l and FIG. 3m. While shorter pixel dwell-times and larger pixel sizes increase writing speed, FIG. 3n-q shows that the required resolution is lost for pixel sizes larger than 17 nm, at a pixel dwell-time of 50 μs, as also confirmed by Fast Fourier Transformation (FFT) analysis.

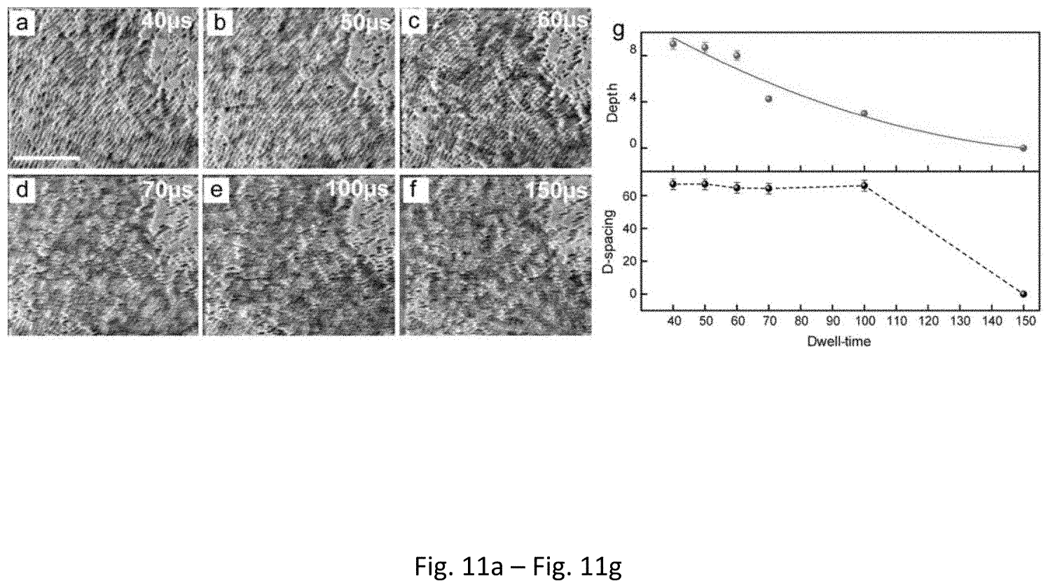

On the other hand, FIG. 11 shows that, fixing the pixel size to 16 nm, a pixel dwell-time of 40 μs gives acceptable patterning resolution. Based on these optimized settings, it is possible to calculate the real throughput for patterning mm² areas by taking into consideration also the extra time required to invert the trajectory of the probe at the end of each patterning line, which is estimated to be 0.05 s per turnaround. We define nₜ as the number of turnarounds per mm². Also, we need to consider the time, tchange, spent to replace each of the thermal probes required to pattern 1 mm², with a total number of thermal probes per mm² equal to n_{probes}. The final formula is the following (Equation 1):

$$\frac{1}{throughput ( {mm}^{2} / h )} = \frac{1 {mm}^{2}}{{d}_{pixel}^{2}} \times {t}_{dwell} + {n}_{t} \times {t}_{turnaround} + {t}_{change} \times {n}_{probes}$$

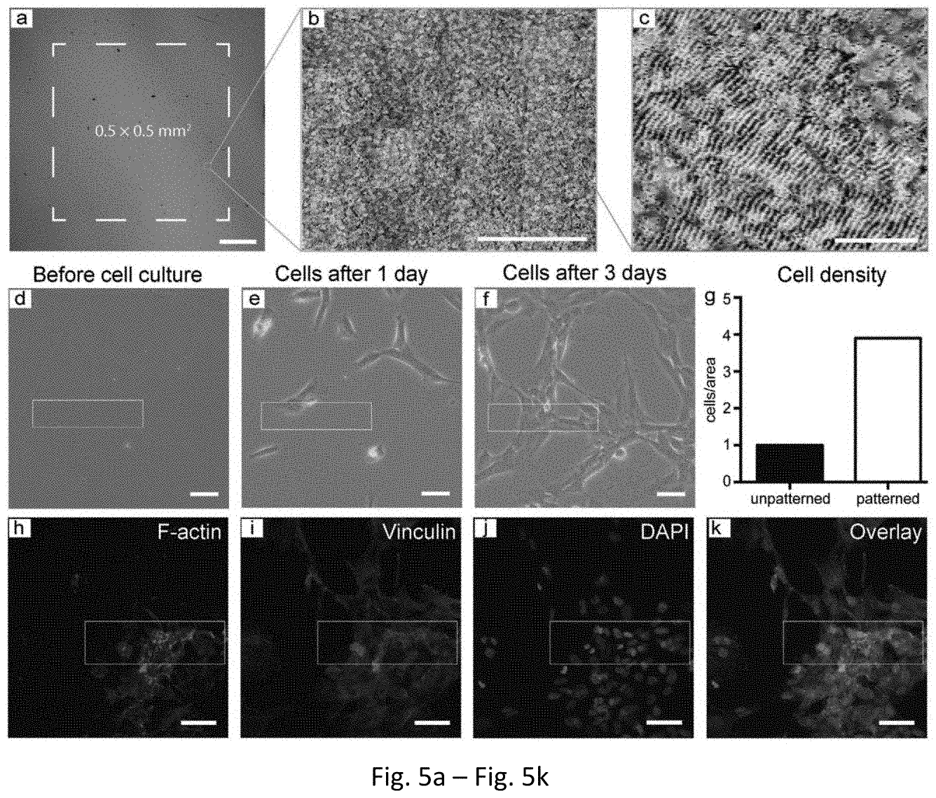

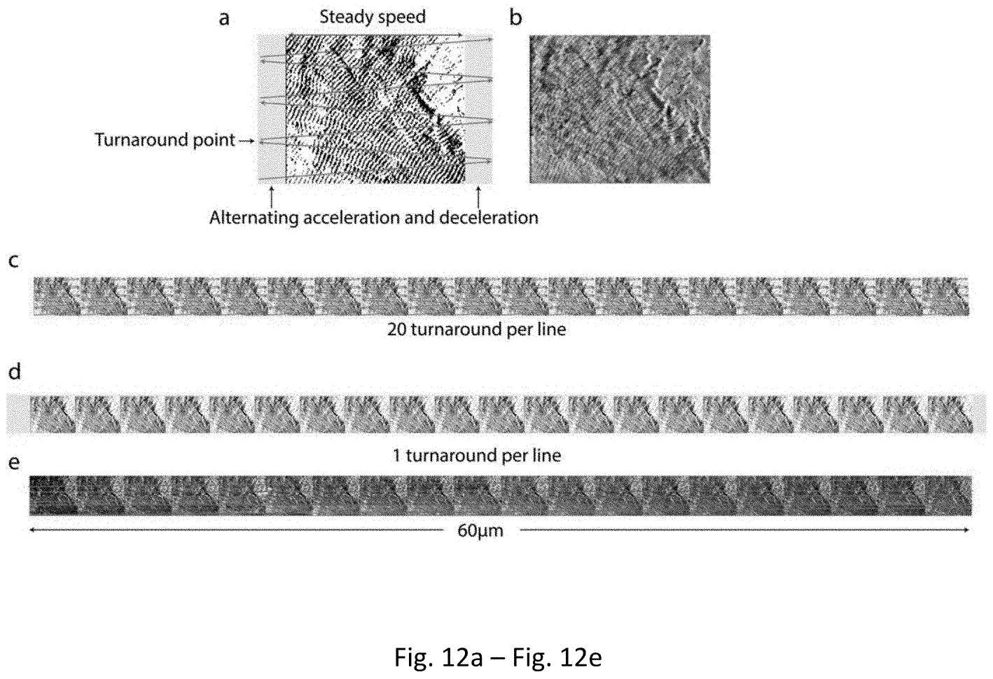

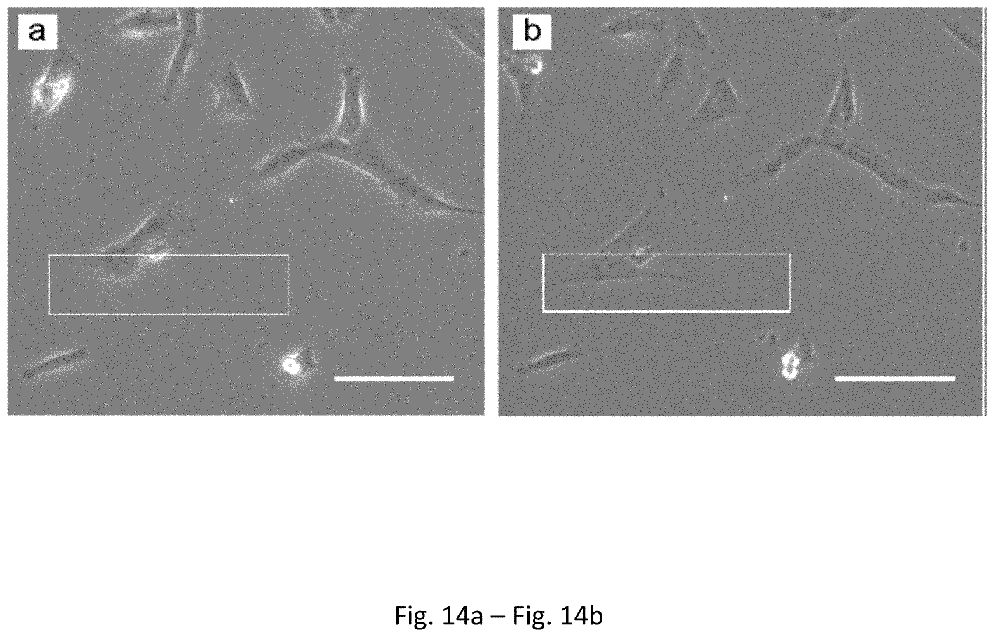

We remark that n_{probes} depends on the writing parameters and also on the polymer resist used for the fabrication. In order to minimize the number of probe turnarounds during writing while preserving the resolution and the fidelity, we perform a series of experiments, as shown in FIG. 12, which demonstrate that a turnaround every 60 μm, corresponding to the maximal extension of the piezo scanner, can still provide excellent sub-15 nm resolution. Finally, by combining all these newly established writing procedures, and using dₚᵢₓₑₗ=16 nm, t_{dwell}=40 μs and a turnaround every 60 μm, we fabricate 0.5 mm×0.5 mm bone tissue replicas (see FIG. 5a-c) in 25 h, at a throughput of ≈0.01 mm² h⁻¹, which is about one or two orders of magnitude faster, for sub-15 nm resolution, compared to previous work (S. T. Zimmermann, et al., ACS Appl. Mater. Interfaces 2017, 9, 41454; S. W. Tang, et al., ACS Appl. Mater. Interfaces 2019, 11, 18988). Using a noncommercial tSPL system, speeds of 0.05 mm² h⁻¹ have been demonstrated for areas smaller than 174 μm² (i.e., for a single probe), and taking into account the turnaround time (P. C. Paul, et al., Nanotechnology 2011, 22, 275306). However, the real throughput to pattern 1 mm² must include the third term in Equation (1), which decreases the writing speed due to probe-change time, depending on the resist material used during the fabrication. On the other hand, here, we demonstrate that to replicate biological tissues instead of requiring about 5550 thermal probes mm⁻² with standard operational modes (using unfiltered input images), we can decrease the number of required probes by a factor of 6 as a result of the thresholding filtering (see FIG. 3), and a further factor of 5±1 as a result of the probe washing (see FIG. 10), which results in a total reduction in costs of about 30 times and a significant increase in throughput. Finally, we demonstrate the applicability of the tSPL bone tissue replicas for stem cell studies (see FIG. 5d-g). In particular, we fabricate a 0.2 mm×0.05 mm bone replica in the PMCC resist spin coated on a thin ITO glass (FIG. 5d), and culture on this sample human iMSCs for 3 d (FIG. 5e,f). The results show that, after seeding, the cells crawl towards the patterned replica and divide on it, forming a multilayer cell system as clearly shown by the higher fluorescence intensity in FIG. 5h-k and FIG. 14. FIG. 5g shows that the number of cells on the patterned area (bone replica) is three time larger than the number of cells on the unpatterned area. Particularly, the cells connect to the patterned replica via the formation of focal adhesion points. All these observations indicate that the bone tissue replicas in PMCC support the growth of human iMSCs.

### Reusability of Bone Tissue Replicas in the PMCC Resist for Repeated Cell Cultures

To further mitigate the problem of high costs and low throughput, we demonstrate that the bone tissue replicas in the PMCC resist on the ITO glass can be reused across several cell culture experiments. To show that the nanopatterns remain unaltered after each cell culture cycle, we pattern several grooves in the PMCC resist, with a length of 1.6 μm, a width of about 90 nm, and a depth of about 12 nm (FIG. 6a), and measure the changes in their dimensions after 3-d cell culture, cell removal, and sample washing cycles (see the Experimental Section and FIG. 6). During all the consecutive cell culture experiments, the PMCC resist adheres well to the ITO glass without any sign of cracking or delamination and the grooves remain unaltered (FIG. 6). To quantitatively evaluate the changes in the grooves with respect to the pristine pattern throughout seven cell culture cycles, we measure the average groove depth, di, over the seven different grooves for each cycle i, and the corresponding average groove full width at half maximum (FWHM) after each experiment (FIG. 6). We observe that throughout the seven cycles of cell culture, cell removal, and sample washing cycles the average change in groove depth and FWHM is less than 5%. Importantly, the cell culture cycles neither affect the nanoscale pattern nor compromise the cell compatible properties of the PMCC resist, as seen in FIG. 6b-h showing that human iMSCs attach, stretch, and grow on the PMCC resist as they do on a conventional cell culture substrate. The ability to reuse the bone tissue replicas on PMCC for repeated cell culture studies represents a time- and cost-effective solution to obviate the limited throughput capabilities of tSPL for biomedical oriented applications. The here demonstrated reusability allows to reuse the same replica for both statistical purposes, and for performing transcriptomic and proteomic analyses, as well as other pertinent molecular assays to study cell response and differentiation.

### Simultaneous Nanopatterning of Bone Tissue Topography and Amine Functionality

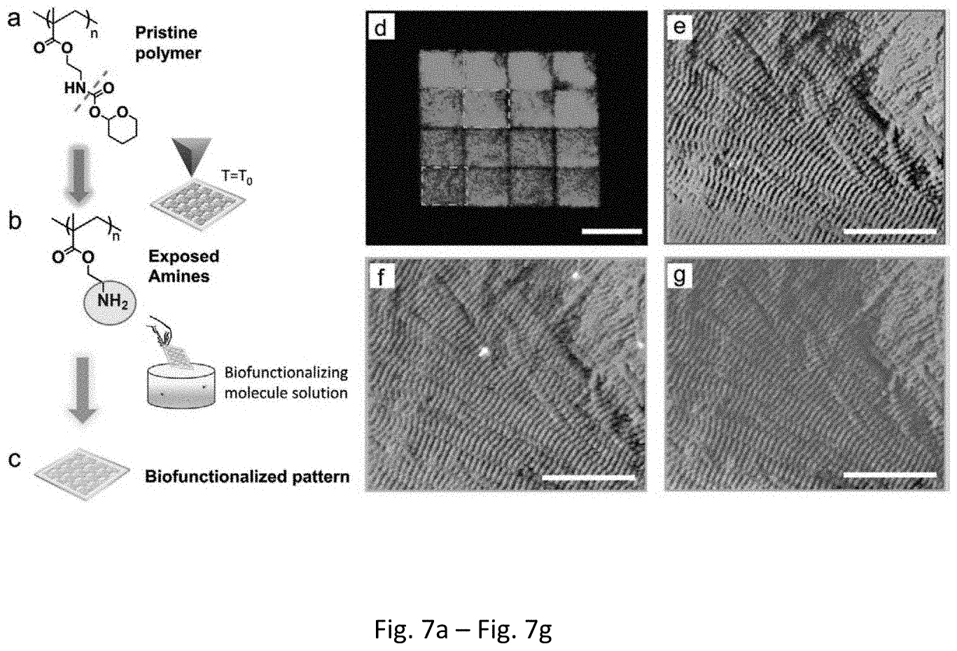

The ability to simultaneously pattern topographical and chemical features of biological tissues creates unprecedented opportunities for the production of biomimetic materials that control cell behavior (H. G. Craighead, et al., Curr. Opin. Solid State Mater. Sci. 2001, 5, 177; J. Yang, et al., Adv. Mater. 2009, 21, 300; G. Dos Reis, et al., Macromol. Biosci. 2010, 10, 842). Recently, we demonstrated the capability of tSPL to simultaneously pattern topography and chemistry with sub-10 nm resolution in the PMCC resist through heat-induced deprotection of amine groups from tetrahydropyran on the carbamate block of the PMCC copolymer (see FIG. 7a-c) (X. Y. Liu, et al., ACS Appl. Mater. Interfaces 2019, 11, 41780; D. B. Wang, et al., Adv. Funct. Mater. 2009, 19, 3696; E. Albisetti, et al., Nanotechnology 2016, 27, 315302; K. M. Carroll, et al., Langmuir 2013, 29, 8675; X. Y. Liu, et al., Faraday Discuss. 2019, 219, 33). Here, we use a similar approach to demonstrate that tSPL can replicate the complex quasi-3D topographical structure of the bone tissue while simultaneously activating amine groups on the surface, which can eventually be functionalized with ad hoc biomolecules, such as tissue-specific molecules (S. J. Bryant, et al., Acta Biomater. 2005, 1, 243). To showcase the capability of tSPL to pattern amine groups with different density on the bone replicas, we fabricate an array of replicas using different writing temperatures (T) and pressures, exploiting the method described in detail in X. Y. Liu, et al., ACS Appl. Mater. Interfaces 2019, 11, 41780 and in the Experimental Section. We then labeled the amine groups on the replicas using an Alexa 488 fluorophore through electrostatic interaction between the negatively-charged sulfonate groups on the Alexa 488 and the positively-charged amine groups (see the Experimental Section for more details) (X. Y. Liu, et al., ACS Appl. Mater. Interfaces 2019, 11, 41780). The different fluorescence intensities of the replicas in each row (FIG. 7d) confirm the presence of amine groups on the bone tissue replicas as well as the ability to control the local amine density by varying the writing T, (see corresponding thermal images of the replicas for selected T (FIG. 7e-g)).

### CONCLUSION

In conclusion, this work introduces bio-tSPL, a fabrication platform that adapts and scales up tSPL for the cost- and time-effective production of mm size replicas of biological tissues on transparent substrates with unprecedented nanoscale control of topography and chemistry over large areas. In particular, we replicate with sub-15 nm resolution the complex quasi-3D morphology of the bone tissue in a biocompatible polymer resist that supports the growth and proliferation of stem cells, providing a biomimetic system for testing and controlling cell behavior. This bio-tSPL platform dramatically reduces the problems of throughput and high cost of tSPL by introducing smart software and post-patterning procedures, as well as a biocompatible, functional polymer resist that withstands multiple cell culture cycles, allowing the reuse of the tissue replicas. The orders of magnitude improvement in costs and throughput, as well as the cell culture compatibility and reusability of the replicas, make bio-tSPL a feasible nanofabrication method for biomedical research. More specifically, the mm-size bone replicas enable the growth of a sufficient number of cells to perform state-of-the-art molecular biology analyses (E. Shapiro, et al., Nat. Rev. Genet. 2013, 14, 618), which can identify microenvironmental cues controlling cell behavior, and for drug screening purposes. Additionally, the achieved costs, patterning speed, and reusability of the tissue replicas allow the testing of an adequate number of samples for statistical analysis. For example, in six months 24 mm-size replicas can be produced, and reused for testing at least five times each, for a total of 120 tests. Therefore, bio-tSPL opens a unique path to generate fundamental understanding of the cell-tissue microenvironment interactions, to test drugs in tissue-mimetic environments, and ultimately to control stem cell fate and tissue regeneration for diverse biomedical applications.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of producing a biological tissue replica comprising:
using a localized source of heat to pattern and replicate the morphology of a biological tissue in a thermosensitive polymer coating a substrate, and wherein the thermosensitive polymer is biocompatible or cell culture compatible, thereby producing the biological tissue replica.

2. The method of claim 1, wherein the method comprises using thermal scanning probe lithography (tSPL) to pattern and replicate the morphology of the biological tissue in the thermosensitive polymer coating the substrate.

3. The method of claim 1, wherein the substrate comprises a solid substrate, combination of materials, or a stacked combination of materials.

4. The method of claim 1, wherein the solid substrate comprises glass, quartz, silicon, metal, or ceramics.

5. The method of claim 1, wherein the thermosensitive polymer has a stiffness that can be tuned by heat or UV light, with values ranging from kPa to GPa.

6. The method of claim 5, wherein the stiffness of the thermosensitive polymer can be spatially patterned with micron scale resolution.

7. The method of claim 1, wherein the thermosensitive polymer is polymethacrylate-carbamate-cinnamate copolymer (PMCC).

8. The method of claim 1, wherein the replicated morphology is further transferred using one or more etching procedures from the thermosensitive polymer to the substrate.

9. The method of claim 1, wherein the substrate is a medical device.

10. The method of claim 9, wherein the medical device is an orthopedic implant or dental implant.

11. The method of claim 1, wherein the biological tissue replica has a nanometer resolution.

12. The method of claim 1, wherein an atomic force microscopy image, scanning electron microscopy image, and/or transmission electron microscopy image of the biological tissue morphology is used as an input for the patterning.

13. The method of claim 12, wherein the image is filtered by setting a threshold value to separate the image into background pixels and foreground pixels, wherein the background pixels are not patterned and wherein the foreground pixels are patterned.

14. The method of claim 2, wherein tSPL is conducted with a dwell time of about 40-70 μs and a pixel size of about 12-20 nm.

15. The method of claim 1, wherein the method comprises exposing amine groups of the polymer and attaching a biomolecule to the exposed amine groups to biofunctionalize the patterned morphology.

16. The method of claim 1, further comprising seeding cells onto the biological tissue replica and culturing the cells in a cell culture media.

17. The method of claim 16, wherein the cells are selected from the group consisting of: stem cells, progenitor cells, mesenchymal stem cells, induced mesenchymal stem cells, induced pluripotent stem cells, embryonic stem cells, osteocytes, osteoblasts, osteoclasts, osteoprogenitor cells, mesenchymal cells, chondrocytes, cartilage progenitor cells, fibroblasts, endothelial cells, myocytes, cardiomyocytes, cardiac progenitor cells myoblasts, skin cells, skin stem cells, and tumor cells.

18. A biological tissue replica comprising a patterned biological tissue morphology on a substrate coated with a thermosensitive polymer to replicate the biological tissue morphology, wherein the thermosensitive polymer is biocompatible or cell culture compatible.

19. The replica of claim 18, wherein the replica is made by using thermal scanning probe lithography (tSPL) to pattern a biological tissue morphology on the substrate.

20. A method of performing an assay comprising:
a) seeding the biological tissue replica of claim 18 with cells;
b) culturing the cells in a cell culture media; and
c) performing an assay using the cultured cells, thereby performing the assay.
